(19) United States
(12) Patent Application Publication
(10) Pub. No.: US 2010/0035907 A1
(43) Pub. Date: Feb. 11, 2010

(54) NEW 2,4-DIANILINOPYRIMIDINES, PREPARATION THEREOF AS DRUGS, PHARMACEUTICAL COMPOSITIONS AND USE THEREOF ESSENTIALLY AS IKK INHIBITORS

(75) Inventors: Monsif BOUABOULA, Juvignac (FR); Michael BOSCH, Marsillargues (FR); Pierre CASELLAS, Castelnau-le-lez (FR); Jean-Flaubert Nguefack, Lattes (FR); Bernard Tonnerre, Vailhauques (FR); Jean Wagnon, Montpellier (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: Sanofi-aventis, Paris (FR)

(21) Appl. No.: 12/495,992

(22) Filed: Jul. 1, 2009

Related U.S. Application Data
(63) Continuation of application No. PCT/FR2008/000001, filed on Jan. 2, 2008.

(30) Foreign Application Priority Data
Jan. 5, 2007 (FR) 0700063

(51) Int. Cl. A61K 31/506; C07D 239/12; C07D 401/12; A61P 35/00; A61P 3/10

(52) U.S. Cl. 514/275; 544/323; 544/324

(57) ABSTRACT

The disclosure relates to a compound of formula (I):

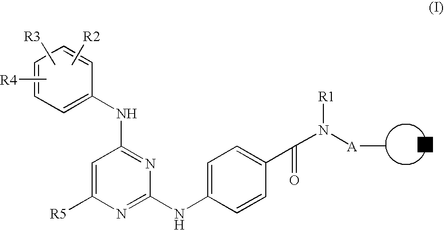

wherein R1, R2, R3, R4, R5, A and Y are as defined in the specification, to compositions containing them, to processes for preparing them, and to their use in the treatment or prevention of conditions capable of being modulated by the inhibition of the activity of protein kinases.

This application is a continuation of International application No. PCT/FR2008/000001, filed Jan. 2, 2008, which is incorporated herein by reference in its entirety; which claims the benefit of priority of French Patent Application No. 0700063, filed Jan. 5, 2007.

The present invention relates to novel 2,4-dianilinopyrimidine derivatives, to their process of preparation, to the novel intermediates obtained, to their application as medicaments, to the pharmaceutical compositions including them and to the novel use of such 2,4-dianilinopyrimidine derivatives.

Patent WO200164654-A1 mentions 2,4-di(hetero)arylpyrimidines substituted in the 5 position which are inhibitors of the kinases CDK 2 and FAK; likewise, other aminopyrimidines which are inhibitors of serine-threonine kinases and of CDK are presented in WO2003030909-A1. Patent WO2004046118-A2 describes 2,4-diphenylaminopyrimidine derivatives as inhibitors of cell proliferation.

A series of 5-cyano-2-aminopyrimidines is presented as inhibitors of the kinases KDR and FGFR in WO200078731-A1, of other pyrimidines as inhibitors of FAK and of IGFR in WO2004080980A-1, and also of ZAP-70, FAK and/or Syk tyrosine kinase in WO2003078404-A1, and of the polo-like kinases PLK in WO2004074244-A2, as cytostatic agents.

Likewise, other patents describe pyrimidines which are inhibitors of reverse transcriptase in the treatment of HIV-related infections (WO200185700-A2, WO200185699-A2, WO200027825A1 and WO2003094920A1).

A subject-matter of the present invention is thus novel 2,4-dianilinopyrimidine derivatives possessing inhibiting effects on protein kinases.

The products of the present invention can thus in particular be used in the prevention or treatment of conditions capable of being regulated by the inhibition of the activity of protein kinases.

Mention is more particularly made, among these protein kinases, of the protein kinase IKK-alpha (IKKα) and IKK-beta (IKKβ).

The compounds of the present invention are kinase inhibitors, in particular inhibitors of IKK-alpha and IKK-beta; consequently, they inhibit NF-KB (nuclear factor kappa B) activity; thus, they can be used in the treatment or prophylaxis of inflammatory diseases, cancer and diabetes.

NF-kB (nuclear factor kappa B) belongs to a family of complexes of transcription factors composed of different combinations of Rel/NF-KB polypeptides. The members of this family of NF-KB-related polypeptides regulate the expression of genes involved in immune and inflammatory responses (Bames P J and Karin M (1997), New Engl. J. Med., 336, 1066-1071, and Baeuerle P A and Baichwal V R (1997), Adv. Immunol., 65, 111-137). Under basal conditions, NF-KB dimers are retained in the inactive form in the cytoplasm by inhibitory proteins which are members of the IKB family (Beg et al., Genes Dev., 7, 2064-2070, 1993; Gilmore and Morin, Trends Genet., 9, 427-433), 1993); Haskil et al., Cell, 65, 1281-1289, 1991). The proteins of the IKB family mask the NF-KB nuclear translocation signal. The stimulation of the cell by various types of ligands, such as cytokines, the anti-CD40 ligand, lipopolysaccharide (LPS), oxidizing agents, mitogens, such as phorbol ester, viruses and many other stimulants, results in the activation of the IKB-kinase (IKK) complex, which in its turn will phosphorylate IKB at serine residues 32 and 34. Once phosphorylated, IKB will be subject to ubiquitinations resulting in its degradation by the proteasome (26S), thus making possible the release and the translocation of NF-KB into the nucleus, where it will become bonded to specific sequences in the promoters of target genes, thus resulting in their transcription.

In the IKB-kinase (IKK) complex, the main kinases are IKK1(IKKα) and IKK2 (IKKβ), which are capable of directly phosphorylating the various classes of IKB. In this IKK complex, IKK2 is the dominant kinase (Mercurio et al., Mol Cell Biol, 19, 1526, 1999-, Zandi et al., Science, 28 1: 1 3) 60, 1998; Lee et al., Proc. Natl. Acad. Sci. USA, 95,93) 19, 1998). Among the genes regulated by NF-KB, many code for proinflammatory mediators, cytokines, cell adhesion molecules, acute phase proteins, which in their turn will also bring about activation of NF-KB by autocrine or paracrine mechanisms.

The inhibition of the activation of NF-KB appears to be very important in the treatment of inflammatory diseases.

In addition, NF-KB plays a role in the growth of normal cells but also of malignant cells.

The proteins produced by the expression of genes regulated by NF-KB comprise cytokines, chemokines, adhesion molecules, mediators of cell growth, of angiogenesis. Furthermore, various studies have shown that NF-KB plays an essential role in neoplastic transformations. For example, NF-KB can be associated with the transformation of cells in vitro and in vivo following overexpression, amplification, rearrangement or translocation events (Mercurio R and Manning A M (1999), Oncogene, 18, 6163-6171). In some human lymphoid tumor cells, the genes coding for the various NF-KB members are rearranged or amplified. It has been shown that NF-KB can promote cell growth by bringing about the transcription of cyclin D, which, associated with the hyperphosphorylation of Rb, results in G1 to S phase transition and inhibition of apoptosis.

It has been shown that, in a large number of tumor cell lines, a constitutive NF-KB activity is found following the activation of IKK2. NF-KB is constitutively activated in Hodgkin's diseases and inhibition of NF-KB blocks the growth of these lymphomas. Moreover, inhibition of NF-KB by the expression of the repressor IKBa results in apoptosis of the cells expressing the oncogenic allele of H-Ras (Baldwin, J. Clin. Invest., 107, 241 (2001), Bargou et al., J. Clin. Invest., 100, 2961 (1997), Mayo et al., Science, 178, 1812 (1997)).

The constitutive NF-KB activity appears to contribute to oncogenesis through the activation of several antiapoptotic genes, such as Al/Bfi-1, IEX-1, MAP, which thus results in the suppression of the cell death pathway. Through the activation of cyclin D, NF-KB can promote the growth of tumor cells. The regulation of adhesion molecules and surface proteases suggests a role of NF-KB signaling in metastases.

NF-KB is involved in the induction of chemoresistance. NF-KB is activated in response to a certain number of chemotherapy treatments. It has been shown that the inhibition of NF-KB by the use of the superrepressor form of IKBa in parallel with the chemotherapy treatment increases the effectiveness of the chemotherapy in xenograft models.

The present invention relates to the products of formula (I):

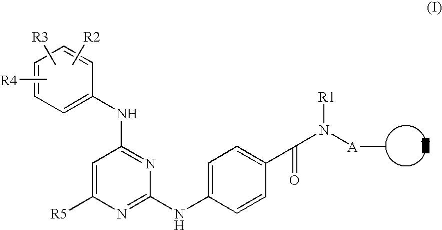

in which:

R2, R3 and R4, which are identical or different, are such that one represents a halogen atom or CF₃ and the other two, which are identical or different, represent a hydrogen atom or a halogen atom or an alkyl radical or an alkoxy radical optionally substituted by one or more halogen atoms;

R5 represents a hydrogen atom or a halogen atom;

R1 represents a hydrogen atom, a cycloalkyl radical or an alkyl, alkenyl or alkynyl radical, all optionally substituted by one or more identical or different radicals chosen from halogen atoms, OR8 and NR8R9, the alkyl radicals represented by R1 in addition optionally being substituted by a saturated or unsaturated 5-membered heterocyclic radical attached via a carbon atom and optionally substituted by one or more radicals chosen from one or more halogen atoms and alkyl or alkoxy radicals;

A represents a single bond or a —CH₂—CO—NR6— radical and R6, which is identical to or different from R1, is chosen from the values of R1;

the ring including Y (or ring(Y)) being monocyclic or bicyclic, having from 4 to 10 ring members and being saturated or partially saturated with Y representing an oxygen atom O, a sulphur atom S, optionally oxidized by one or two oxygen atoms, or a radical chosen from N—R7, C═O or its dioxolane as protective group for the carbonyl functional group, CF₂, CH—OR8 or CH—NR8R9;

it being understood that the ring including Y (or ring(Y)), when Y represents NR7, can include a carbon bridge composed of 1 to 3 carbons,

R7 represents the hydrogen atom, a cycloalkyl radical or an alkyl, CH₂-alkenyl or CH₂-alkynyl radical, all optionally substituted by a naphthyl radical or by one or more identical or different radicals chosen from halogen atoms and hydroxyl, alkoxy, phenyl and heteroaryl radicals, the alkyl radicals represented by R7 in addition being optionally substituted by a hydroxyl, —NR8R9, —CO—NR8R9, phosphonate, alkylthio, optionally oxidized to give sulphone, or optionally substituted heterocycloalkyl radical;

R8 represents the hydrogen atom or alkyl, cycloalkyl or heterocycloalkyl radicals, themselves optionally substituted by one or more radicals chosen from halogen atoms and hydroxyl, alkoxy, NH₂, NHalkyl, N(alkyl)₂, —CONH₂, —CONHalkyl or —CON(alkyl)₂ radicals, the alkyl radicals represented by R8 in addition being optionally substituted by an alkylthio radical, by an optionally substituted phenyl radical or by a saturated or unsaturated, optionally substituted, heterocyclic radical;

NR8R9 is such that either R8 and R9, which are identical or different, are chosen from the values of R8 or R8 and R9 form, with the nitrogen atom to which they are bonded, a cyclic amine which can optionally include one or two other heteroatoms chosen from O, S, N or NR10, the cyclic amine thus formed being itself optionally substituted;

all the above heterocyclic, heterocycloalkyl and heteroaryl radicals being composed of 4 to 10 ring members (unless specified) and including 1 to 4 heteroatoms chosen, if appropriate, from O, optionally oxidized S, N and NR10;

all the above naphthyl, phenyl, heterocyclic, heterocycloalkyl and heteroaryl radicals and also the cyclic amine which can be formed by R8 and R9 with the nitrogen atom to which they are bonded being themselves optionally substituted by one or more identical or different radicals chosen from halogen atoms and hydroxyl, alkoxy, alkyl, hydroxyalkyl, alkoxyalkyl, CN, CF₃, NH₂, NHalk or N(alk)₂ radicals;

R10 represents a hydrogen atom or an alkyl radical,

the said products of the formula (I) being in all the possible isomeric forms, racemic, enantiomeric and diastereoisomeric, and also the addition salts with inorganic and organic acids of the said products of formula (I).

Mention may particularly be made, among the products of formula (I) as defined above in which R1, R2, R3, R4, R5 and A have the meanings indicated above, of those in which the ring(Y) is chosen from the following definitions:
when ring(Y) is such that Y represents C—OH, CF₂, CH—OR8 or CH—NR8R9, the ring formed can in particular be a cyclobutyl, cyclopentyl, cyclohexyl or cycloheptyl and particularly a cyclohexyl, these radicals thus being substituted, in particular in the para position, respectively by OH, 2F, the OR8 radical or the NR8R9 radical in which R8 and R9 are chosen from the meanings defined above;
when ring(Y) is such that Y represents NR7, the ring formed can in particular be an azetidinyl, pyrrolidinyl or piperidinyl radical with the nitrogen atom N in the para or in the meta position, which thus carries the substituent R7 as defined above. When ring(Y) such that Y represents NR7 includes a carbon bridge composed of 1 to 3 carbons, the ring formed can in particular be the 8-azabicyclo[3.2.1]octan-3-yl ring or also a ring chosen from the following rings: N,9-dimethyl-9-azabicyclo[3.3.1]nonan-3-yl, N,6-dimethyl-6-azabicyclo[3.2.1]octan-3-yl, N,3-dimethyl-3-azabicyclo[3.2.1]octan-8-yl or also N,3-dimethyl-3-azabicyclo[3.3.1]nonan-9-yl;
when ring(Y) is such that Y represents NR7, the ring formed can in particular be a bicyclic radical, such as, for example, quinolinyl or indolizinyl;
when ring(Y) is such that Y represents S, the ring formed can in particular be a tetrahydrothiopyranyl or a tetrahydrothiophenyl: when ring(Y) is such that Y represents SO₂, the ring formed can in particular be a dioxidotetrahydro-3-thiophenyl;
when ring(Y) is such that Y represents O, the ring formed can in particular be a tetrahydrofuran or tetrahydropyran. When ring(Y) is such that Y represents the dioxolane of C═O, the ring formed can in particular be dioxaspiro[4.5]dec-8-yl.

The present invention relates in particular to the products of formula (I) as defined above in which R2, R3, R4, R5, A and ring(Y) have the meanings indicated above and R1 represents a hydrogen atom or a linear or branched alkyl radical including from 1 to 5 carbon atoms or else R1 represents this alkyl radical substituted by a saturated or unsaturated heterocycle, preferably monocyclic with 5 ring members, itself optionally substituted as indicated above,

the said products of formula (I) being in all the possible isomeric forms, racemic, enantiomeric and diastereoisomeric, and also the addition salts with inorganic and organic acids of the said products of formula (I).

The present invention relates in particular to the products of formula (I) as defined above in which R2, R3, R4, R5 and A have the meanings indicated above, R1 represents a hydrogen atom or an optionally substituted, linear or branched, alkyl radical including from 1 to 4 carbon atoms and in particular CH₃ and ring(Y) is such that Y represents NR7 with R7 representing a linear or branched alkyl radical including from 1 to 6 carbon atoms which is optionally substituted by a radical chosen from hydroxyl, CF₃, phosphonate, sulphone, phenyl and saturated or unsaturated, monocyclic or bicyclic, heterocyclic radicals, these phenyl and heterocyclic radicals themselves optionally being substituted as indicated above,

the said products of formula (I) being in all the possible isomeric forms, racemic, enantiomeric and diastereoisomeric, and also the addition salts with inorganic and organic acids of the said products of formula (I).

The present invention relates very particularly to the products of formula (I) as defined above in which R2, R3, R4, R5 and A have the meanings indicated above,

R1 represents a linear or branched alkyl radical including from 1 to 4 carbon atoms and in particular CH₃ and ring(Y) is such that Y represents NR8R9 in which R8 represents a hydrogen atom or CH₃ and R9 represents a linear or branched alkyl radical including from 1 to 6 carbon atoms which is optionally substituted by a radical chosen from hydroxyl, CF₃, phosphonate, sulphone, phenyl and saturated or unsaturated, monocyclic or bicyclic, heterocyclic radicals, these phenyl and heterocyclic radicals being themselves optionally substituted as indicated above,

the said products of formula (I) being in all the possible isomeric forms, racemic, enantiomeric and diastereoisomeric, and also the addition salts with inorganic and organic acids of the said products of formula (I).

The present invention thus relates in particular to the products of formula (I) as defined above in which R2, R3, R4, R5 and A are chosen from the meanings indicated above and the other substituents are chosen from preferred values defined as follows:
R1 represents a hydrogen atom, a CH₃ radical or a linear or branched alkyl radical including from 1 to 4 carbon atoms which is optionally substituted by an NH₂, NHalk or N(alk)₂ radical or by a saturated or unsaturated heterocycle, preferably a monocycle comprising 5 or 6 ring members, which ring members are as defined above and optionally substituted as indicated above or below, and ring(Y) represents a piperidinyl or pyrrolidinyl substituted on its nitrogen atom by R7, which represents an alkyl radical optionally substituted by a hydroxyl, —NR8R9, —CO—NR8R9, phosphonate or alkylthio, optionally oxidized to sulphone, radical;
R1 is chosen from the values defined above and ring(Y) represents a cyclohexyl radical substituted by an NR8R9 radical as defined above;
R1 represents a CH₃ radical optionally substituted by a saturated or unsaturated heterocycle as defined above and R7 represents a CH₃ radical;
R1 represents a hydrogen atom or a CH₃ radical and ring(Y) represents a piperidinyl or an 8-azabicyclo[3.2.1]octan-3-yl ring substituted on their nitrogen atom by R7 with R7 as defined above.

Mention may be made, among the products of formula (I) as defined above in which R1, R2, R3, R4, R5 and A have the meanings indicated above, for example, of those in which ring(Y) is chosen from the following definitions:
ring(Y) such that Y represents —N—R7 with R7 representing H;
ring(Y) such that Y represents —N—R7 with R7 representing CH₃;
ring(Y) such that Y represents —N—R7 with R7 representing cycloalkyl, such as, in particular, cyclopropyl;
ring(Y) such that Y represents —N—R7 with R7 representing an alkyl radical, in particular a CH₃, C₂H₅ or C₃H₇ radical, substituted by a phosphonate;
ring(Y) such that Y represents —N—R7 with R7 representing an alkyl radical, in particular a CH₃, C₂H₅ or C₃H₇ radical, substituted by an alkylthio, such as S—CH₃ or S—C₂H₅, with S optionally oxidized to sulphone to form, for example, SO₂—CH₃ or SO₂—C₂H₅;
ring(Y) such that Y represents —N—R7 with R7 representing alkyl, such as, in particular, CH₃ or C₂H₅, substituted by one or more radicals chosen from halogen atoms, such as, in particular, F, and phenyl and mono- or bicyclic heterocycle radicals, phenyl and heterocycle themselves optionally substituted by one or more radicals chosen from halogen atoms and alkyl, alkoxy, OH, CN, CF₃, NH₂, NHalk and N(alk)₂ radicals: mention may in particular be made, among these heterocycles carried by R7, of unsaturated 5-membered heterocycles including one to four heteroatoms chosen from N, O and S: thus, R7 can represent in particular the —CH₂-thiophenyl, —CH₂-thiazolyl (N,S), —CH₂-thiadiazolyl (N,N,S), —CH₂-furanyl (O), —CH₂-pyrazolyl (N,N), —CH₂-isoxazolyl (N,O) or —CH₂-pyrrolyl (NH, NCH₃) radicals, these radicals, in particular pyrazolyl, isoxazolyl, pyrrolyl or tetrazolyl, being themselves optionally substituted, in particular by alkyl including from 1 to 3 carbon atoms, such as, in particular, CH₃ or C₂H₅;

R7 can also carry heterocycles as defined above, such as the pyridinyl (with N of the pyridine at 3 different positions); 2,3-dihydro-1H-indolyl; quinolyl; isoquinolyl; pyrimidinyl; 2,3-dihydrobenzofuranyl; 1,8-naphthyridinyl; pyridinyl N-oxide; or 4-benzo[1,2,5]oxadiazolyl radicals;
ring(Y) such that Y represents CH—NR8R9 with NR8R9 such that R8 represents a hydrogen atom or an alkyl radical, such as, in particular, CH₃, and R9 represents a linear or branched alkyl radical, such as, in particular, CH₃, C₂H₅ or —CH₂— or —CH(CH₃)— or —CH(CH₃)—CH₂—, substituted either by an optionally substituted, saturated or unsaturated, mono- or bicyclic heterocycle or by an optionally substituted phenyl radical. Mention may in particular be made, among the heterocycles carried by R9, of the following radicals: pyridinyl (with N of the pyridine at 3 different positions); 2,3-dihydro-1H-indolyl; quinolyl; isoquinolyl; pyrimidinyl; 2,3-dihydrobenzofuranyl; 1,8-naphthyridinyl; 4-benzo[2,1,3]oxadiazolyl; or benzo[2,1,3]thiadiazolyl;

such heterocycles are optionally substituted by one or more radicals as defined above or below.

Mention may be made, among the products of formula (I) as defined above in which R2, R3, R4, R5, A and the ring(Y) have the meanings indicated above, for example, of those in which R1 is chosen from the following definitions:
R1 represents H;
R1 represents CH₃;
R1 represents alkenyl (3C) radicals, such as allyl, or alkynyl (3C) radicals, such as propargyl;
R1 represents alkyl and in particular CH₃, C₂H₅, C₃H₇, substituted by one or more identical or different radicals chosen from halogen atoms and NH₂, NH(alk), N(alk)₂, NH—CH₂—CH₂OH, NH—CH₂—C₃H₇—OH, NH(CH₂—CF₃), alkoxy or OH radicals or a saturated heterocycle, such as, for example, pyrrolidinyl, piperidinyl, morpholinyl or tetrahydrofuranyl, or an unsaturated heterocycle, such as, in particular, those defined above for R7.

A subject-matter of the present invention is thus the products of the formula (I) as defined above in which:

R2, R3 and R4, which are identical or different, are such that one represents a halogen atom or CF₃ and the other two, which are identical or different, represent a hydrogen atom, a halogen atom or an alkyl or alkoxy radical optionally substituted by one or more halogen atoms;

R5 represents a hydrogen atom or a halogen atom;

R1 represents a hydrogen atom, a cycloalkyl radical or an alkyl, alkenyl or alkynyl radical, all optionally substituted by one or more identical or different radicals chosen from halogen atoms, OR8 and NR8R9;

A represents a single bond or a —CH₂—CO—NR6— radical and R6, which is identical to or different from R1, is chosen from the values of R1;

the ring including Y (or ring(Y)) being monocyclic or bicyclic, having from 4 to 10 ring members and being saturated or partially saturated with Y representing an oxygen atom O, a sulphur atom S, optionally oxidized by one or two oxygen atoms, or a radical chosen from N—R7, C═O, CF₂, CH—OR8 or CH—NR8R9;

R7 represents a hydrogen atom or an alkyl, CH₂-alkenyl or CH₂-alkynyl radical, all optionally substituted by a naphthyl radical or by one or more identical or different radicals chosen from halogen atoms and hydroxyl, phenyl and heteroaryl radicals, all of these naphthyl, phenyl and heteroaryl radicals being themselves optionally substituted; the heteroaryl radicals being composed of 5 to 10 ring members and including 1 to 4 heteroatoms chosen from O, S, N and NR10;

R8 represents the hydrogen atom or alkyl, cycloalkyl or heterocycloalkyl radicals, themselves optionally substituted by one or more radicals chosen from halogen atoms and hydroxyl, alkoxy, NH₂, NHalkyl or N(alkyl)₂ radicals;

NR8R9 is such that either R8 and R9, which are identical or different, are chosen from the values of R8 or R8 and R9 form, with the nitrogen atom to which they are bonded, a cyclic amine which can optionally include one or two other heteroatoms chosen from O, S, N or optionally substituted NR10;

R10 represents a hydrogen atom or an alkyl radical;

all the naphthyl, phenyl and heteroaryl radicals and also the cyclic amine which can be formed by R8 and R9 with the nitrogen atom to which they are bonded being themselves optionally substituted by one or more identical or different radicals chosen from halogen atoms and hydroxyl, alkoxy, alkyl, hydroxyalkyl, alkoxyalkyl, CF₃, NH₂, NHalk or N(alk)₂ radicals; the said products of formula (I) being in all the possible isomeric forms, racemic, enantiomeric and diastereoisomeric, and also the addition salts with inorganic and organic acids of the said products of formula (I).

In the products of formula (I) and in that which follows, the terms indicated have the meanings which follow:
the term “halogen” denotes the fluorine, chlorine, bromine or iodine atoms and preferably the fluorine, chlorine or bromine atoms;
the term “alkyl radical” denotes a linear or branched radical including at most 6 carbon atoms and in particular the methyl, ethyl, propyl, isopropyl, n-butyl, isobutyl, sec-butyl, tert-butyl, pentyl, isopentyl, sec-pentyl, tert-pentyl, neopentyl, hexyl, isohexyl, sec-hexyl or tert-hexyl radicals and also their linear or branched positional isomers;
the term “hydroxyalkyl radical” denotes the alkyl radicals indicated above substituted by one or more hydroxyl radicals;
the term “alkenyl radical” denotes a linear or branched radical including at most 6 carbon atoms and preferably 4 carbon atoms chosen, for example, from the following values: ethenyl or vinyl, propenyl or allyl, 1-propenyl, n-butenyl, isobutenyl, 3-methylbut-2-enyl, n-pentenyl or hexenyl, and also their linear or branched positional isomers: mention is more particularly made, among the alkenyl values, of the allyl or butenyl values;
the term “alkynyl radical” denotes a linear or branched radical including at most 6 carbon atoms and preferably 4 carbon atoms chosen, for example, from the following values: ethynyl, propynyl or propargyl, butynyl, n-butynyl, isobutynyl, 3-methylbut-2-ynyl, pentynyl or hexynyl, and also their linear or branched positional isomers: mention is more particularly made, among the alkynyl values, of the propargyl value;
the term “alkylene radical” denotes a linear or branched divalent radical including at most 6 carbon atoms resulting from the above alkyl radical and thus chosen, for example, from the methylene, ethylene, propylene, isopropylene, butylene, isobutylene, sec-butylene or pentylene radicals;
the term “alkoxy radical” denotes a linear or branched radical including at most 6 carbon atoms chosen, for example, from among methoxy, ethoxy, propoxy, isopropoxy, butoxy, linear, secondary or tertiary, pentoxy, hexoxy and heptoxy, and also their linear or branched positional isomers;
the term “cycloalkyl radical” denotes a monocyclic or bicyclic carbocyclic radical including from 3 to 7 ring members and denotes in particular the cyclopropyl, cyclobutyl, cyclopentyl, cyclohexyl and cycloheptyl radicals;
the term “aryl radical” denotes unsaturated carbocyclic radicals which are monocyclic or composed of fused rings.

Mention may in particular be made, as examples of such aryl radicals, of the phenyl or naphthyl radicals;
the term “heterocyclic radical” denotes a saturated carbocyclic (heterocycloalkyl) radical or a partially or completely unsaturated (heteroaryl) carbocyclic radical composed of 4 to 10 ring members interrupted by one or three identical or different heteroatoms chosen from the oxygen, nitrogen or sulphur atoms:
   mention may in particular be made, among 5-membered heteroaryl radicals, of the radicals including one to four heteroatoms chosen from N, optionally oxidized, O and S, optionally oxidized, mention may be made of radicals 2-thiophenyl, 3-thiophenyl, dioxidothienyl, -thiazolyl (N,S), -furyl (O), 2-furyl, pyrrolyl (NH, NCH₃), isothiazolyl, diazolyl, thiadiazolyl (N,N,S), 1,3,4-thiadiazolyl, oxazolyl, oxadiazolyl, isoxazolyl (N,O), 3-isoxazolyl, 4-isoxazolyl, imidazolyl or pyrazolyl (N,N), triazolyl or tetrazolyl groups and more particularly the oxazolyl, isoxazolyl (N,O) or pyrazolyl radicals; all these rings optionally being substituted by one or more radicals as defined above or below, these substituents, of course, being located at the positions chemically acceptable for each of these rings;
   mention may in particular be made, among 6-membered heteroaryl radicals, of the pyridyl, such as 2-pyridyl, 3-pyridyl and 4-pyridyl, pyridyl N-oxide, pyrimidinyl, pyridazinyl and pyrazinyl radicals;
   mention may be made, among fused heteroaryl radicals comprising at least one heteroatom chosen from sulphur, nitrogen and oxygen, for example, of the benzothiophenyl, benzofuryl, benzoxazolyl, indazolyl, indolyl, indolinyl, indolinonyl, quinolyl, isoquinolyl, azaindolyl, benzimidazolyl, benzothiazolyl, naphthyridinyl, such as 1,8-naphthyridinyl, imidazo[4,5]pyridinyl, indolizinyl, quinazolinyl, 2,3-dihydro-1H-indolyl, 2,3-dihydrobenzofuranyl or 4-benzo[1,2,5]oxadiazolyl radicals;
   mention may more particularly be made, among fused heterocyclyl radicals, of the benzothiophenyl, benzofuranyl, benzodihydrofuranyl, indolyl, indolinyl, indolinonyl, benzimidazolyl, benzothiazolyl, benzoxodiazolyl, benzothiodiazolyl, naphthyridinyl, indazolyl, quinolyl, such as 4-quinolyl or 5-quinolyl, isoquinolyl, azaindolyl, such as 4-azaindolyl or 3-azaindolyl, imidazo[4,5]pyridyl, indolizinyl or quinazolinyl radicals;
   mention may be made, as heterocycloalkyl (saturated), for example, of the oxiranyl, oxetanyl, tetrahydrofuranyl, dioxolanyl, dithiolanyl, tetrahydropyranyl, dioxanyl, aziridinyl, azetidinyl, pyrrolidinyl, piperidinyl, azepinyl, diazepinyl, piperazinyl, morpholinyl, thiomorpholinyl, dioxidomorpholinyl or imidazolidinyl radicals; mention may more particularly be made of the pyrrolidinyl, piperidinyl, azepinyl, piperazinyl or morpholinyl radicals;
   all the cyclic radicals being optionally substituted as indicated above or below;
the terms “alkylamino or NH(alk) radical” and “dialkylamino or N(alk)₂ radical” thus denote amino NH₂ radicals respectively substituted by one or two linear or branched alkyl radicals, identical or different in the case of dialkylamino, chosen from the alkyl radicals as defined above and optionally substituted as indicated above or below: mention may be made, for example, of the methylamino, ethylamino, propylamino or butylamino radicals or the dimethylamino, diethylamino or methylethylamino radicals;
the term “cycloalkylamino radical” thus denotes an amino radical substituted in particular by a cycloalkyl radical chosen from the radicals defined above: mention may thus be made, for example, of the cyclopropylamino, cyclobutylamino, cyclopentylamino or also cyclohexylamino radicals;
the term “cyclic amine” denotes a monocyclic or bicyclic radical including from 3 to 10 ring members in which at least one carbon atom is replaced by a nitrogen atom, it being possible for this cyclic radical also to include one or more other heteroatoms chosen from O, S, SO₂, N or NR10 with R10 as defined above: mention may be made, as examples of such cyclic amines, for example, of the pyrrolyl, piperidyl, morpholinyl, piperazinyl, pyrrolidinyl or azetidinyl radicals. Mention may more particularly be made of the piperidinyl, morpholinyl, piperazinyl or azetidinyl radicals.

The term “patient” denotes human beings but also other mammals. The term “prodrug” denotes a product which can be converted in vivo by metabolic mechanisms (such as hydrolysis) to a product of formula (I). For example, an ester of a product of formula (I) comprising a hydroxyl group can be converted by hydrolysis in vivo to its mother molecule.

Mention may be made, as examples of esters of products of formula (I) comprising a hydroxyl group, such as of the acetates, citrates, lactates, tartrates, malonates, oxalates, salicylates, propionates, succinates, fumarates, maleates, methylenebis(β-hydroxynaphthoates), gentisates, isethionates, di(p-toluoyl)tartrates, methanesulphonates, ethanesulphonates, benzenesulphonates, p-toluenesulphonates, cyclohexylsulfamates and quinates.

Esters of products of formula (I) which are particularly useful comprising a hydroxyl group can be prepared from acid residues, such as those described by Bundgaard et al., J. Med. Chem., 1989, 32, page 2503-2507: these esters include in particular (aminomethyl)benzoates which are substituted, dialkylaminomethylbenzoates in which the two alkyl groups can be bonded together or can be interrupted by an oxygen atom or by an optionally substituted nitrogen atom or an alkylated nitrogen atom or also (morpholinomethyl)benzoates, e.g. 3- or 4-(morpholinomethyl)benzoates, and (4-alkylpiperazin-1-yl)benzoates, e.g. 3- or 4-(4-alkylpiperazin-1-yl)benzoates. When the products of formula (I) comprise an amino radical which can be salified by an acid, it is clearly understood that these acid salts also form part of the invention. Mention may be made, for example, of the salts provided with hydrochloric acid or methanesulphonic acid.

The addition salts with inorganic or organic acids of the products of formula (I) can, for example, be the salts formed with hydrochloric, hydrobromic, hydriodic, nitric, sulphuric, phosphoric, propionic, acetic, trifluoroacetic, formic, benzoic, maleic, fumaric, succinic, tartaric, citric, oxalic, glyoxylic, aspartic or ascorbic acids, alkylmonosulphonic acids, such as, for example, methanesulphonic acid, ethanesulphonic acid or propanesulphonic acid, alkyldisulphonic acids, such as, for example, methanedisulphonic acid or α,β-ethanedisulphonic acid, arylmonosulphonic acids, such as benzosulphonic acid, and aryldisulphonic acids.

It may be remembered that stereoisomerism may be defined in its broad sense as the isomerism of compounds having the same expanded formulae but the various groups of which are positioned differently in space, such as, in particular, in monosubstituted cyclohexanes, the substituent of which can be in the axial or equatorial position. However, there exists another type of stereoisomerism due to the different spatial arrangements of fixed substituents, either on double bonds or on rings, which are often referred to as E/Z geometrical isomerism or cis-trans isomerism or diastereoisomerism. The term “stereoisomer” is used in the present patent application in its broadest sense and thus relates to all the compounds indicated above.

The present invention has in particular as subject-matter the products of formula (I) as defined above in which:

R2, R3 and R4, which are identical or different, are such that one represents a fluorine or chlorine atom or CF₃ and the other two, which are identical or different, represent a hydrogen atom, a fluorine or chlorine atom or a methyl or methoxy radical optionally substituted by one or more fluorine atoms;

R5 represents a hydrogen atom or a fluorine or chlorine atom;

R1 represents a hydrogen atom, a cycloalkyl radical or an alkyl radical optionally substituted by one or more identical or different radicals chosen from the fluorine atom, OR8 and NR8R9;

A represents a single bond or a —CH₂—CO—NR6— radical and R6 represents a hydrogen atom or a linear or branched alkyl radical including at most 4 carbon atoms;

the ring including Y (or ring(Y)) being monocyclic or bicyclic, having from 4 to 10 ring members and being saturated or partially saturated with Y representing an oxygen atom O, a sulphur atom S, optionally oxidized by one or two oxygen atoms, or a radical chosen from N—R7, C═O, CF₂, CH—OR8 or CH—NR8R9;

R7 represents a hydrogen atom or an alkyl radical optionally substituted by one or more identical or different radicals chosen from halogen atoms and phenyl and heteroaryl radicals, the phenyl and heteroaryl radicals being themselves optionally substituted by one or more identical or different radicals chosen from halogen atoms and hydroxyl, alkoxy, alkyl, hydroxyalkyl, alkoxyalkyl, CF₃, NH₂, NHalk or N(alk)₂ radicals; the heteroaryl radicals being composed of 5 to 7 ring members and including 1 to 3 heteroatoms chosen from O, S, N and NR10;

R8 represents the hydrogen atom, linear or branched alkyl radicals including at most 4 carbon atoms or cycloalkyl radicals including from 3 to 6 ring members, the alkyl and cycloalkyl radicals being themselves optionally substituted by a hydroxyl radical;

NR8R9 is such that either R8 and R9, which are identical or different, are chosen from the values of R8 or R8 and R9 form, with the nitrogen atom to which they are bonded, a cyclic amine chosen from the pyrrolyl, piperidyl, morpholinyl, pyrrolidinyl, azetidinyl and piperazinyl radicals, the piperazinyl radical optionally being substituted on its second nitrogen atom by an alkyl radical;

the said products of formula (I) being in all the possible isomeric forms, racemic, enantiomeric and diastereoisomeric, and also the addition salts with inorganic and organic acids of the said products of formula (I).

In particular, the ring including Y can be composed of 4 to 7 ring members and can be saturated with Y representing an oxygen atom O, a sulphur atom S, optionally oxidized by one or two oxygen atoms, or a radical chosen from N—R7, CH—NH₂, CH—NHalk or CH—N(alk)₂, with R7 as defined above or below.

The present invention has in particular as subject-matter the products of formula (I) as defined above in which:

R2, R3 and R4, which are identical or different, are such that one represents a fluorine atom or CF₃ and the other two represent one a hydrogen atom and the other a fluorine or chlorine atom or a methyl radical;

R5 represents a hydrogen atom or a chlorine atom;

R1 represents a hydrogen atom or a cyclopropyl, methyl, ethyl, propyl or butyl radical optionally substituted by one or more identical or different radicals chosen from the fluorine atom and the hydroxyl, amino, alkylamino, dialkylamino, piperidinyl, morpholinyl, azetidinyl, piperazinyl, pyrrolidinyl and pyrrolyl radicals;

A represents a single bond, —CH₂—CO—NH— or —CH₂—CO—NCH₃— and the ring including Y is chosen from the cyclohexyl radical, itself optionally substituted by amino; the tetrahydropyranyl radical; the dioxidothiophenyl radical; and the pyrrolidinyl, piperidinyl, azepinyl, indolizinyl and quinazolinyl radicals optionally substituted by one or more identical or different radicals chosen from the methyl, propyl, butyl, isopropyl, isobutyl, isopentyl or ethyl radicals, themselves optionally substituted by one or more radicals chosen from halogen atoms and the following radicals: hydroxyl, phenyl, itself optionally substituted by one or more halogen atoms, quinolyl, pyridyl, optionally oxidized on its nitrogen atom, thiophenyl, thiazolyl, thiadiazolyl, tetrazolyl, pyrazinyl, furyl and imidazolyl, itself optionally substituted by alkyl;

the said products of formula (I) being in all the possible isomeric forms, racemic, enantiomeric and diastereoisomeric, and also the addition salts with inorganic and organic acids of the said products of formula (I).

The present invention has in particular as subject-matter the products of formula (I) as defined above in which:

R2, R3 and R4, which are identical or different, are such that one represents a fluorine atom or CF₃ and the other two represent one a hydrogen atom and the other a fluorine or chlorine atom or a methyl radical;

R5 represents a hydrogen atom;

R1 represents a methyl radical or an ethyl radical optionally substituted by an amino, alkylamino, dialkylamino or pyrrolidinyl radical;

A represents a single bond and the ring including Y represents a cyclohexyl radical itself optionally substituted by amino or a piperidinyl or pyrrolidinyl radical optionally substituted on its nitrogen atom by a methyl, propyl, butyl, isopropyl, isobutyl, isopentyl or ethyl radical, themselves optionally substituted by one or more halogen atoms or a radical chosen from hydroxyl; thiadiazolyl; tetrazolyl; phenyl, itself optionally substituted by halogen; quinolyl; pyridyl, optionally oxidized on its nitrogen atom; furyl; and imidazolyl, itself optionally substituted by alkyl;

the said products of formula (I) being in all the possible isomeric forms, racemic, enantiomeric and diastereoisomeric, and also the addition salts with inorganic and organic acids of the said products of formula (I).

Mention is made in particular of the products of formula (I) in which A represents a single bond, the other substituents R1, R2, R3, R4, R5 and ring(Y) of the said products of formula (I) being chosen from the values indicated above.

Mention is thus particularly made of the products of formula (I) in which R5 represents a hydrogen atom, the other substituents R1, R2, R3, R4, A and ring(Y) of the said products of formula (I) being chosen from the values indicated above.

Preference is given to the products of formula (I) as defined above in which, when NR8R9 does not form a cyclic amine, then NR8R9 is such that R8 represents a hydrogen atom or an alkyl radical and R9 is chosen from all the values defined for R8.

When one of R2, R3 and R4 represents alkoxy, methoxy is preferred.

Products of formula (I) as defined above in which:

R2, R3 and R4, which are identical or different, are such that one represents a fluorine atom or CF₃ and the other two represent one a hydrogen atom and the other a fluorine or chlorine atom or a methyl radical;

R5 represents a hydrogen atom;

R1 represents a hydrogen atom or a methyl radical;

A represents a single bond and the ring including Y is chosen from the tetrahydropyranyl or dioxidothiophenyl radicals and the pyrrolidinyl, piperidinyl and azepinyl radicals optionally substituted on their nitrogen atoms (in the 2 or 3 position of the ring) by a methyl, ethyl, propyl or butyl radical, themselves optionally substituted by one or more halogen atoms or a phenyl, pyridyl, thiophenyl, thiazolyl, thiadiazolyl, pyrazinyl, furyl or imidazolyl radical;

the said products of formula (I) being in all the possible isomeric forms, racemic, enantiomeric and diastereoisomeric, and also the addition salts with inorganic and organic acids of the said products of formula (I).

The present invention has particularly as subject-matter the products of formula (I) corresponding to the following names:
4-[4-(4-Fluoro-3-methylphenylamino)pyrimidin-2-ylamino]-N-methyl-N-(1-methylpiperidin-4-yl)benzamide;
4-[4-(4-Fluoro-3-methylphenylamino)pyrimidin-2-ylamino]-N-methyl-N-[1-(4,4,4-trifluorobutyl)piperidin-3-yl]benzamide;
4-[4-(4-Fluoro-3-methylphenylamino)pyrimidin-2-ylamino]-N-methyl-N-[1-(1,2,3-thiadiazol-4-ylmethyl)piperidin-3-yl]benzamide;
N-Methyl-N-(1-methylpiperidin-4-yl)-4-[4-(4-(trifluoromethyl)phenylamino)pyrimidin-2-ylamino]benzamide;
N-Methyl-N-(tetrahydropyran-4-yl)-4-[4-(4-(trifluoromethyl)phenylamino)pyrimidin-2-ylamino]benzamide;
N-(1-Methylpiperidin-4-yl)-N-[2-(pyrrolidin-1-yl)ethyl]-4-[4-(4-(trifluoromethyl)phenylamino)pyrimidin-2-ylamino]benzamide; and
4-({4-[(4-Fluorophenyl)amino]pyrimidin-2-yl}amino)-N-(octahydroindolinizin-7-yl)benzamide;
   the said products of formula (I) being in all the possible isomeric forms, racemic, enantiomeric and diastereoisomeric, and also the addition salts with inorganic and organic acids of the said products of formula (I).

The present invention additionally has as subject-matter the processes for the preparation of the products of formula (I) as defined above.

The present invention has in particular as subject-matter the process for the preparation of the products of formula (I) as defined above, characterized in that the product of formula (II):

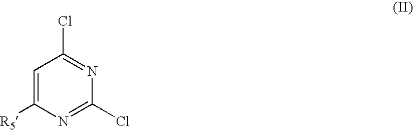

in which R₅′ has the meaning indicated above for R5 in which the possible reactive functional groups are optionally protected,
is reacted with a product of formula (III):

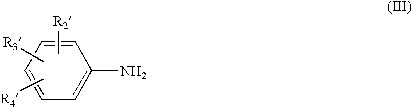

in which R₂′, R₃′ and R₄′ have the meanings indicated above for R2, R3 and R4 respectively in which the possible reactive functional groups are optionally protected,
in order to obtain a product of formula (IV):

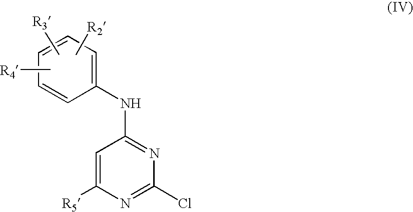

in which R₂′, R₃′, R₄′ and R₅′ have the meanings indicated above,
which product of formula (IV) as defined above is reacted with the methyl ester of 4-aminobenzoic acid of formula (V), in order to obtain the product of formula (VI):

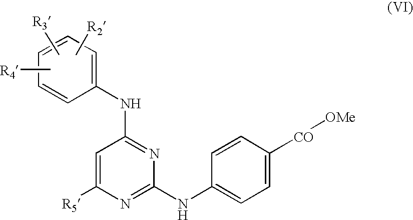

in which R₂′, R₃′, R₄′ and R₅′ have the meanings indicated above, which product of formula (VI) is saponified to give its corresponding acid of formula (VII):

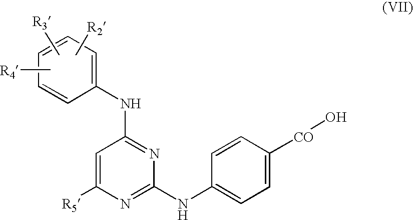

in which R₂′, R₃′, R₄′ and R₅′ have the meanings indicated above,
which product of formula (VII) is reacted with an amine of formula (VIII):

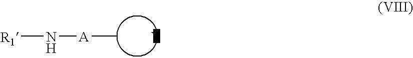

in which R₁′ has the meaning indicated above for R1 in which the possible reactive functional groups are optionally protected by protective groups,
in order to obtain a product of formula (I₁):

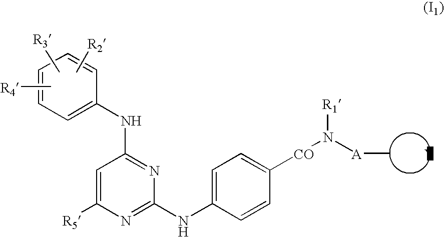

in which R₁′, R₂′, R₃′, R₄′ and R₅′ have the meanings indicated above,
which products of formula (I₁) can be products of formula (I) and which, in order to obtain products or other products of formula (I), can be subjected, if desired and if necessary, to one or more of the following conversion reactions, in any order:
a) an oxidation reaction on an alkylthio group to give the corresponding sulphoxide or sulphone,
b) a conversion reaction on an alkoxy functional group to give a hydroxyl functional group or also on a hydroxyl functional group to give an alkoxy functional group,
c) an oxidation reaction on an alcohol functional group to give an aldehyde or ketone functional group,
d) an elimination reaction on the protective groups which can be carried by the protected reactive functional groups,
e) a salification reaction by an inorganic or organic acid in order to obtain the corresponding salt,
f) a resolution reaction on the racemic forms to give resolved products,
the said products of formula (I) thus obtained being in all the possible isomeric forms, racemic, enantiomeric and diastereoisomeric.

The present invention also has as subject-matter a process for the preparation of the products of formula (I) as defined above in which Y represents the NR7 radical as defined above with R7 representing CH₂—RZ and RZ representing an alkyl, alkenyl or alkynyl radical, all optionally substituted as indicated above and in particular by a naphthyl radical or by one or more identical or different radicals chosen from halogen atoms and phenyl and heteroaryl radicals, all these naphthyl, phenyl and heteroaryl radicals being themselves optionally substituted by one or more identical or different radicals chosen from halogen atoms and the hydroxyl, alkoxy, alkyl, hydroxyalkyl, alkoxyalkyl, CF₃, NH₂, NHalk or N(alk)₂ radicals.

Such a process is characterized in that the compound of formula (A):

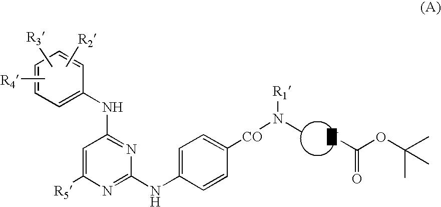

in which R₁′, R₂′, R₃′, R₄′, R₅′ and ring(N) have the meanings indicated above,
is subjected to a deprotection reaction on the carbamate functional group, in order to obtain a product of formula (IX):

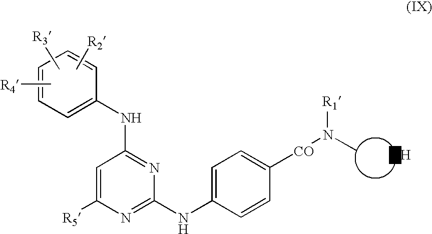

in which R₁′, R₂′, R₃′, R₄′, R₅′ and ring(N) have the meanings indicated above,
which product of formula (IX) is subjected to reductive amination conditions
in the presence of the aldehyde or ketone of formula (X):

RZ′—CR8′O (X)

in which RZ′ has the meaning indicated above and represents an optionally substituted alkyl, alkenyl or alkynyl radical as indicated in any one of the preceding claims and in which the possible reactive functional groups are optionally protected by protective groups,
and R8′ has the meaning indicated above for R8 in which the possible reactive functional groups are optionally protected by protective groups,
in order to obtain a product of formula (I₂):

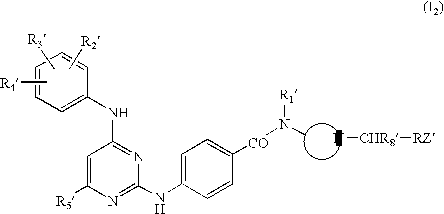

in which R₁′, R₂′, R₃′, R₄′, R₅′, ring(N), RZ′ and R8′ have the meanings indicated above,
which products of formula (I₂) can be products of formula (I) and which, in order to obtain products or other products of formula (I), can be subjected, if desired and if necessary, in any order, to one or more of the conversion reactions a) to f) as defined above,
the said products of formula (I₂) thus obtained being in all the possible isomeric forms, racemic, enantiomeric and diastereoisomeric.

Under preferred conditions for implementing the invention, the processes described above can be carried out in the following way:

The product of formula (II) is subjected to the action of the product of formula (III) as defined above, in particular in an alcohol, such as, for example, butanol, propanol or ethanol, or dimethylformamide, between 80 and 140° C., to give a product of formula (IV) as defined above.

The product of formula (IV) as defined above is subjected to the action of a methyl ester of 4-aminobenzoic acid of formula (V), in particular in an alcohol, such as butanol, at a temperature of 100 to 140° C., in order to give the product of formula (VI) as defined above.

This product of formula (VI) is saponified to give its corresponding acid of formula (VII) while proceeding according to the normal methods known to a person skilled in the art, such as, in particular, by the action of sodium hydroxide or potassium hydroxide in water.

The product of formula (VII) thus obtained is reacted with the amine of formula (VIII) defined above according to the coupling methods known to a person skilled in the art, such as, for example, by amide coupling in the presence of a coupling agent, such as BOP, DCC or TBTU, in a solvent, such as, for example, dimethylformamide or dichloromethane, in order to give a product of formula (I₁) as defined above.

The deprotection reaction on the carbamate functional group of the compound of formula (A) in order to obtain a product of formula (IX) can be carried out using, for example, an acid agent, such as pure trifluoroacetic acid at a temperature of approximately 0° C. or a mixture of this acid with an appropriate solvent, such as methylene chloride, at approximately 0° C., are also using hydrochloric acid in solution in ether or dioxane at a temperature of between 0° C. and ambient temperature.

The product of formula (IX) is subjected to reductive amination conditions in the presence of the aldehyde or ketone of formula (X), in order to give a product of formula (I₂) as defined above, for example with sodium borocyanide or sodium triacetoxyborohydride, in a solvent, such as methanol, tetrahydrofuran (THF) or their mixture, as a medium with a pH between 4 and 7.

Depending on the values of R₁′, R2, R3, R4 and R5, and RZ′, the products of formulae (I₁) and (I₂) as defined above can thus constitute products of formula (I) as defined above or can be converted to products of formula (I) by the usual methods known to a person skilled in the art, for example by being subjected to one or more of the reactions a) to f) indicated above.

Furthermore, it may be noted that such reactions a) to f) for the conversion of substituents into other substituents can also be carried out on the starting materials and on the intermediates as defined above before continuing the synthesis according to the reactions indicated in the above processes.

The various reactive functional groups which may be carried by some compounds of the reactions defined above can, if necessary, be protected: this concerns, for example, hydroxyl, acyl or also amino and monoalkylamino radicals, which can be protected by appropriate protective groups.

The following non-exhaustive list of examples of the protection of reactive functional groups may be mentioned:
hydroxyl groups can be protected, for example, by alkyl radicals, such as tert-butyl, trimethylsilyl, tert-butyldimethylsilyl, methoxymethyl, tetrahydropyranyl, benzyl or acetyl,
amino groups can be protected, for example, by acetyl, trityl, benzyl, tert-butoxycarbonyl, benzyloxycarbonyl or phthalimido radicals or other radicals known in the chemistry of peptides and can then be released under the usual conditions known to a person skilled in the art.

The reactions to which the products of formula (I′) as defined above can be subjected, if desired or if necessary, can be carried out, for example, as indicated below.

The saponification reactions can be carried out according to the usual methods known to a person skilled in the art, such as, for example, in a solvent, such as methanol or ethanol, dioxane or dimethoxyethane, in the presence of sodium hydroxide or potassium hydroxide.

The reduction or oxidation reactions can be carried out according to the usual methods known to a person skilled in the art, such as, for example, in a solvent, such as ethyl ether or tetrahydrofuran, in the presence of sodium borohydride or lithium aluminum hydride; or, for example, in a solvent, such as acetone or tetrahydrofuran, in the presence of potassium permanganate or pyridinium chlorochromate.

a) the possible alkylthio groups of the products described above can, if desired, be converted to the corresponding sulphoxide or sulphone functional groups under the usual conditions known to a person skilled in the art, such as, for example, with peracids, such as, for example, peracetic acid or meta-chloroperbenzoic acid, or also with oxone, sodium periodate, in a solvent, such as, for example, methylene chloride or dioxane, at ambient temperature.

The production of the sulphoxide functional group can be promoted by an equimolar mixture of the product including an alkylthio group and of the reactant, such as, in particular, a peracid.

The production of the sulphone functional group can be promoted by a mixture of the product including an alkylthio group with an excess of the reactant, such as, in particular, a peracid.

b) The possible alkoxy functional groups, such as, in particular, methoxy functional groups, of the products described above can, if desired, be converted to a hydroxyl functional group under the usual conditions known to a person skilled in the art, for example with boron tribromide, in a solvent, such as, for example, methylene chloride, with pyridine hydrobromide or hydrochloride or also with hydrobromic or hydrochloric acid in water or trifluoroacetic acid at reflux.
c) The possible alcohol functional groups of the products described above can, if desired, be converted to an aldehyde or ketone functional group by oxidation under the usual conditions known to a person skilled in the art, such as, for example, by the action of manganese oxide, in order to obtain aldehydes, or by the action of potassium permanganate or pyridinium chlorochromate, in order to access ketones.
d) The elimination of protective groups, such as, for example, those indicated above, can be carried out under the usual conditions known to a person skilled in the art, in particular by acid hydrolysis carried out with an acid, such as hydrochloric, benzenesulphonic, para-toluenesulphonic, formic or trifluoroacetic acid, or also by catalytic hydrogenation. The phthalimido group can in particular be eliminated with hydrazine.

A list of various protective groups which can be used will be found, for example, in Patent BF 2 499 995.

e) The products described above can, if desired, form the subject of salification reactions, for example with an inorganic or organic acid, according to the usual methods known to a person skilled in the art.
f) The possible optically active forms of the products described above can be prepared by resolution of the racemates according to the usual methods known to a person skilled in the art.

Illustrations of such reactions defined above are given in the preparation of the examples described below.

The starting materials of formulae (II), (III) and (VIII) may be known, may be obtained commercially or may be prepared according to the usual methods known to a person skilled in the art, in particular from commercial products, for example by subjecting them to one or more reactions known to a person skilled in the art, such as, for example, reactions described above in a) to f).

The materials of formula (II), which are thus pyrimidine derivatives, and the materials of formula (III), which are aniline derivatives, can be commercial products, such as, for example, dichloropyrimidine, trichloropyrimidine, 4-fluoroaniline, 3,4-difluoroaniline, 4-fluoro-3-chloroaniline or aniline.

The anilines of formula (III) can in particular be commercial anilines, such as, for example, the following trihalogenated anilines:
3,4,5-trifluoroaniline
2,3,4-trifluoroaniline
2-chloro-4,6-difluoroaniline
2,4,5-trifluoroaniline
3-chloro-2,4-difluoroaniline
2,4-dichloro-5-fluoroaniline
4-trifluoromethylaniline.

The amines of formula (VIII) can also be commercially available, such as, for example, methyl(1-methylpiperidin-4-yl)amine.

The amines of formula (VIII) which are not commercially available can be prepared according to methods known to a person skilled in the art.

It may be indicated that, in order to obtain products of formula (I) as defined above in which R1, R2, R3, R4, R5 and A have the meanings indicated above and ring(Y) is such that Y represents NR7 and includes a carbon bridge composed of 1 to 3 carbons, use may be made, as starting materials, of bicyclic amines which can be obtained from commercial compounds, such as tropinone or pseudopelletierine, according to the following references:
Tetrahedron, 2002, 58, 5669-5674
J. Org. Chem., 1996, 61, 3849-3862
J. Med. Chem., 1993, 36, 3703-3720
J. Chem. Soc. Perkin Trans. 1, 1991, 1375-1381
J. Med. Chem., 1994, 37, 2831-2840

Mention may be made, by way of examples, of the following compounds:

N,9-dimethyl-9-azabicyclo[3.3.1]nonan-3-amine

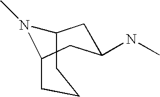

N,6-dimethyl-6-azabicyclo[3.2.1]octan-3-amine

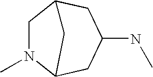

N,3-dimethyl-3-azabicyclo[3.2.1]octan-8-amine

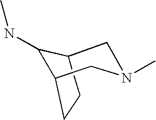

N,3-dimethyl-3-azabicyclo[3.3.1]nonan-9-amine

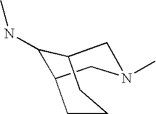

Examples of aldehydes and of ketones of formula (X) are given in the experimental part as non-limiting examples.

The present invention also relates to the process according to Scheme 1 below for the preparation of products of formula (I) as defined above:

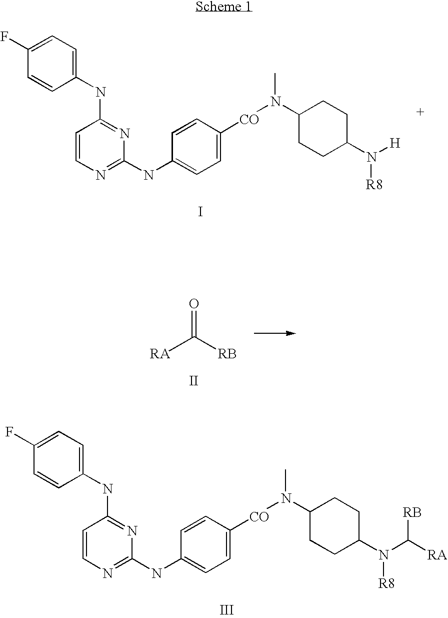

In such a Scheme 1, the NR8-CH(RA)(RB) radical represents certain values of NR8R9 as defined above with R8 as defined above and R9 representing —CH(RA) (RB), that is to say, as defined for R9, a linear or branched alkyl radical optionally substituted by one or more radicals chosen from halogen atoms and hydroxyl, alkoxy, NH₂, NHalkyl, N(alkyl)₂, alkylthio, phenyl and saturated or unsaturated heterocycle radicals, the phenyl and heterocycle radicals being themselves optionally substituted as indicated above.

In particular, RA can represent the hydrogen atom or CH₃ and RB can represent (CH₂)ₙ-A with A representing an optionally substituted heterocycle or phenyl radical as defined above and n representing an integer from 0 to 5.

The stages of the synthetic process of Scheme 1 above can be carried out according to the usual methods known to a person skilled in the art.

The present invention also relates to the process according to Scheme 2 below for the preparation of products of formula (I) as defined above:

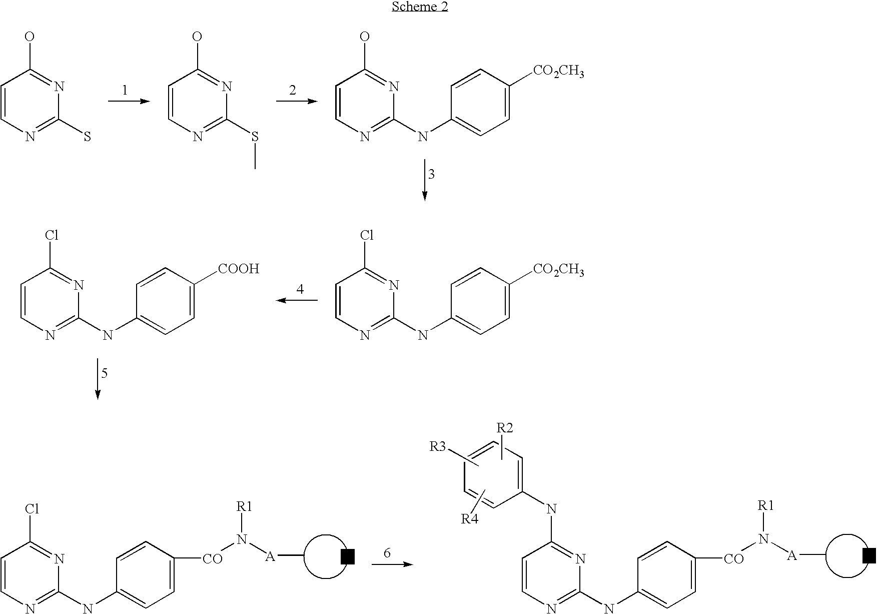

In such a Scheme 2, R1, R2, R3, R4, A and ring(Y) have the meanings indicated above for the products of formula (I).

The stages of the synthetic process of Scheme 2 above can be carried out by using the methyl ester of the aniline in stage 2 and the aniline substituted by R₂′, R₃′ or R₄′ in stage 6 and by making use of the usual methods known to a person skilled in the art or as described in the present invention.

The experimental part below gives non-limiting examples of the preparation of products of formula (I) according to the present invention and also non-limiting examples of starting materials used in these preparations.

Finally, the present invention has as subject-matter some compounds of formulae (A), (IX), (VI) and (VII) as novel industrial products.

The products of formula (I) as defined above and their addition salts with acids exhibit advantageous pharmacological properties.

The compounds of the present invention can thus inhibit the activity of kinases, in particular IKK1 and IKK2, with an IC₅₀ of less than 10 μM.

The compounds of the present invention can thus inhibit the activation of NF-KB and the production of cytokines with IC₅₀ values of less than 10 μM.

The compounds of the present invention can thus inhibit the proliferation of a large sample group of tumor cells with IC₅₀ values of less than 10 μM.

The compounds of the formula (I) can thus have a medicament activity, in particular as inhibitors of IKK1 and IKK2, and can be used in the prevention or treatment of diseases in which inhibition of IKK1 or IKK2 is beneficial, for example the prevention or treatment of diseases such as inflammatory diseases or diseases with an inflammatory component, such as, for example, inflammatory arthritis, including rheumatoid arthritis, osteoarthritis, spondylitis, Reiter's syndrome, psoriatic arthritis, bone resorption diseases; multiple sclerosis, inflammatory diseases of the intestines, including Crohn's disease; asthma, chronic obstructive pulmonary disease, emphysema, rhinitis, acquired myasthenia gravis, Graves' disease, graft rejection, psoriasis, dermatitis, allergic disorders, diseases of the immune system, cachexia, severe acute respiratory syndrome, septic shock, cardiac insufficiency, myocardial infarction, atherosclerosis, reperfusion injuries, AIDs, cancers and disorders characterized by resistance to insulin, such as diabetes, hyperglycaemia, hyperinsulinaemia, dyslipidaemia, obesity, polycystic ovarian disease, hypertension, cardiovascular disorders, syndrome X, autoimmune diseases, such as in particular systemic lupus, lupus erythematosus, glomerulonephritis induced by deficiencies in the immune system, autoimmune insulin-dependent diabetes, retinitis pigmentosa, aspirin-sensitive rhinosinusitis.

The products of formula (I) according to the present invention as modulators of apoptosis can be of use in the treatment of various human diseases including aberrations in apoptosis, such as cancers: such as in particular but without implied limitation follicular lymphomas, carcinomas with p53 mutations, hormone-dependent tumors of the breast, prostate and ovary, and precancerous lesions, such as familial adenomatous polyposis, viral infections (such as in particular but without implied limitation those caused by herpes virus, poxvirus, Epstein-Barr virus, Sindbis virus and adenovirus), myelodysplastic syndromes, ischemic disorders associated with myocardial infarction, cerebral congestion, arrhythmia, atherosclerosis, liver disorders induced by toxins or alcohol, hematological disorders, such as in particular but without implied limitation chronic anemia and aplastic anemia, degenerative diseases of the musculoskeletal system, such as in particular but without implied limitation osteoporosis, cystic fibrosis, diseases of the kidneys and cancers.

It thus appears that the compounds according to the invention have an anticancer activity and an activity in the treatment of other proliferative diseases, such as psoriasis, restenosis, atherosclerosis, AIDs, for example, and also in diseases caused by the proliferation of vascular smooth muscle cells of angiogenesis and then rheumatoid arthritis, neurofibromatosis, atherosclerosis, pulmonary fibrosis, restenosis following angioplasty or vascular surgery, the formation of hypertrophic scars, angiogenesis and endotoxic shock.

These medicaments are employed therapeutically, in particular in the treatment or prevention of diseases caused or exacerbated by the proliferation of cells and in particular tumor cells.

As inhibitor of the proliferation of tumor cells, these compounds are of use in the prevention and treatment of leukemia, solid tumors, both primary and metastatic, carcinomas and cancers, in particular: breast cancer, lung cancer, small intestine cancer, colon and rectal cancer, cancer of the respiratory tract, oropharynx and hypopharynx, esophageal cancer, liver cancer, stomach cancer, bile duct cancer, gall bladder cancer, pancreatic cancer, cancers of the urinary tract, including kidney, urothelium and bladder, cancers of the female genital tract, including cancer of the uterus, cervix or ovaries, choriocarcinoma and trophoblastomic cancer; cancers of the male genital tract, including cancer of the prostate, seminal vesicles or testicles, and germ cell tumors; cancers of the endocrine glands, including cancer of the thyroid, pituitary gland or adrenal glands; cancers of the skin, including hemangiomas, melanomas or sarcomas, including Kaposi's sarcoma; tumors of the brain, nerves, eyes or meninges, including astrocytomas, gliomas, glioblastomas, retinoblastomas, neurinomas, neuroblastomas, schwannomas or meningiomas; hematopoietic malignant tumors; leukemias, such as acute lymphocytic leukemia, acute myeloid leukemia, chronic myeloid leukemia, chronic lymphocytic leukemia, chloromas, plasmacytomas, T- or B-cell leukemias, non-Hodgkin's or Hodgkin's lymphomas, myelomas, various malignant haemopathies. The present invention has in particular as subject-matter the combinations defined as follows.

According to the present invention, the compound or compounds of formula (I) can be administered in combination with one (or more) anticancer active principle(s), in particular antitumor compounds, such as alkylating agents, such as alkylsulphonates (busulfan), dacarbazine, procarbazine, nitrogen mustards (chlormethine, melphalan, chlorambucil), cyclophosphamide or ifosfamide; nitrosoureas, such as carmustine, lomustine, semustine or streptozocin; antineoplastic alkaloids, such as vincristine or vinblastine; taxanes, such as paclitaxel or taxotere; antineoplastic antibiotics, such as actinomycin; intercalating agents, antineoplastic antimetabolites, folate antagonists or methotrexate; purine synthesis inhibitors; purine analogues, such as mercaptopurine or 6-thioguanine; pyrimidine synthesis inhibitors, aromatase inhibitors, capecitabine or pyrimidine analogues, such as fluorouracil, gemcitabine, cytarabine and cytosine arabinoside; brequinar; topoisomerase inhibitors, such as camptothecin or etoposide; anticancer hormonal agonists and antagonists, including tamoxifen; kinase inhibitors, imatinib; growth factor inhibitors; antiinflammatories, such as pentosan polysulphate, corticosteroids, prednisone or dexamethasone; antitopoisomerases, such as etoposide, anthracyclines, including doxorubicin, bleomycin, mitomycin and mithramycin; anticancer metal complexes, platinum complexes, cisplatin, carboplatin or oxaliplatin; interferon-alpha, triphenyl thiophosphoramide or altretamine; antiangiogenic agents; thalidomide; immunotherapy adjuvants; or vaccines.

According to the present invention, the compounds of formula (I) can also be administered in combination with one or more other active principles of use in one of the pathologies indicated above, for example an agent for combating vomiting, pain, inflammation or cachexia.

A subject-matter of the present invention is thus, as medicaments, the products of formula (I) as defined above and also the addition salts with pharmaceutically acceptable inorganic and organic acids of the said products of formula (I).

A subject-matter of the present invention is in particular, as medicaments, the products of formula (I) as defined above having the following names:
4-[4-(4-Fluoro-3-methylphenylamino)pyrimidin-2-ylamino]-N-methyl-N-(1-methylpiperidin-4-yl)benzamide;
4-[4-(4-Fluoro-3-methylphenylamino)pyrimidin-2-ylamino]-N-methyl-N-[1-(4,4,4-trifluorobutyl)piperidin-3-yl]benzamide;
4-[4-(4-Fluoro-3-methylphenylamino)pyrimidin-2-ylamino]-N-methyl-N-[1-(1,2,3-thiadiazol-4-ylmethyl)piperidin-3-yl]benzamide;
N-Methyl-N-(1-methylpiperidin-4-yl)-4-[4-(4-(trifluoromethyl)phenylamino)pyrimidin-2-ylamino]benzamide;
N-Methyl-N-(tetrahydropyran-4-yl)-4-[4-(4-(trifluoromethyl)phenylamino)pyrimidin-2-ylamino]benzamide;
N-(1-Methylpiperidin-4-yl)-N-[2-(pyrrolidin-1-yl)ethyl]-4-[4-(4-(trifluoromethyl)phenylamino)pyrimidin-2-ylamino]benzamide; and
4-({4-[(4-Fluorophenyl)amino]pyrimidin-2-yl}amino)-N-(octahydroindolinizin-7-yl)benzamide;
   and also the addition salts with pharmaceutically acceptable inorganic and organic acids of the said products of formula (I).

Another subject-matter of the present invention is the pharmaceutical compositions comprising, as active principle, at least one of the products of formula (I) as defined above or a pharmaceutically acceptable salt of this product or a prodrug of this product and a pharmaceutically acceptable vehicle.

A subject-matter of the present invention is in particular the use of the products of formula (I) as defined above or of pharmaceutically acceptable salts of these products in the preparation of a medicament intended for the treatment or prevention of a disease by inhibition of the activity of the protein kinase IKK.

A subject-matter of the present invention is thus the use as defined above in which the protein kinase is in a mammal.

A subject-matter of the present invention is thus the use of a product of formula (I) as defined above in the preparation of a medicament intended for the treatment or prevention of a disease chosen from the diseases indicated above.

A subject-matter of the present invention is in particular the use of a product of formula (I) as defined above in the preparation of a medicament intended for the treatment or the prevention of a disease chosen from the following group: inflammatory diseases, diabetes and cancers.

A subject-matter of the present invention is in particular the use of a product of formula (I) as defined above in the preparation of a medicament intended for the treatment or prevention of inflammatory diseases.

A subject-matter of the present invention is in particular the use of a product of formula (I) as defined above in the preparation of a medicament intended for the treatment or prevention of diabetes.

A subject-matter of the present invention is in particular the use of a product of formula (I) as defined above in the preparation of a medicament intended for the treatment of cancers.

A subject-matter of the present invention is in particular the use of a product of formula (I) as defined above intended for the treatment of solid or nonsolid tumors.

A subject-matter of the present invention is in particular the use of a product of formula (I) as defined above intended for the treatment of cancers which are resistant to cytotoxic agents.

A subject-matter of the present invention is in particular the use of a product of formula (I) as defined above in the preparation of medicaments intended for cancer chemotherapy.

A subject-matter of the present invention is in particular the use of a product of formula (I) as defined above in the preparation of medicaments intended for cancer chemotherapy, alone or in combination or in the form of an association as defined above.

A subject-matter of the present invention is in particular the use of a product of formula (I) as defined above as IKK inhibitors.

The present invention relates very particularly to the products of formula (I) as defined above which constitute Examples 1 to 6 of the present invention.

The following examples illustrate the invention without, however, limiting it.

### EXPERIMENTAL PART

### Procedure 1:

### Preparation of 4-[4-(4-fluoro-3-methylphenylamino)pyrimidin-2-ylamino]benzoic Acid

### Stage 1:

### (2-Chloropyrimidin-4-yl)(4-fluoro-3-methylphenyl)amine

5.3 g of 4-fluoro-3-methylphenylamine and then 7 ml of diisopropylethylamine are added with stirring to a mixture comprising 6.3 g of dichloropyrimidine in 100 ml of n-butanol. The reaction mixture is brought to reflux with stirring for 2 hours. The reaction medium is cooled and concentrated to dryness. A K₂CO₃ solution is added to the residue, extraction is carried out three times with ethyl acetate, the combined organic extracts are washed with a saturated NaCl solution and dried over Na₂SO₄, and the crude reaction product is purified by chromatography on a silica column (DCM and then 30% AcOEt in DCM). 3.8 g of the expected product are obtained (melting point=130-131° C.).

### Stage 2:

### 4-[4-(4-Fluoro-3-methylphenylamino)pyrimidin-2-ylamino]benzoic Acid Methyl Ester

A mixture comprising 8 g of chloropyrimidine obtained in stage 1 and 5.1 g of methyl 4-aminobenzoate in n-butanol is heated overnight at 140° C. After cooling, the precipitate is filtered off. This precipitate is washed with Et₂O and is recrystallized from a DCM/MeOH/iPr₂O mixture. 10.5 g of the expected product are thus obtained.

### Stage 3:

### 4-[4-(4-fluoro-3-methylphenylamino)pyrimidin-2-ylamino]benzoic Acid

2.08 g of the product obtained in stage 2 are brought, in the presence of 410 mg of sodium hydroxide in a mixture of MeOH (5 ml), water (5 ml) and dioxane (20 ml), to a temperature of 40° C. overnight. The reaction medium is concentrated to dryness and the residue is taken up in 100 ml of water. The impurities are extracted with two volumes of Et₂O and then the aqueous phase is acidified to pH 6 with 1N HCl. The precipitate formed is filtered off, rinsed with distilled water and suspended in DCM, and the solvent is evaporated. 1.3 g of the expected acid are obtained.

### Procedure 2:

### 4-[4-(4-(Trifluoromethyl)phenylamino)pyrimidin-2-ylamino]benzoic Acid

### Stage 1:

### (2-Chloropyrimidin-4-yl)-(4-(trifluoromethyl)phenyl)amine

In the same way as in Example 1 of Procedure 1, starting with 15 g of dichloropyrimidine in 200 ml of n-butanol, 16 g of 4-(trifluoromethyl)phenylamine and then 18 ml of diisopropylethylamine are added with stirring. The reaction mixture is brought to reflux overnight with stirring. The reaction medium is cooled and concentrated to dryness. A K₂CO₃ solution is added to the residue, extraction is carried out 3 times with ethyl acetate, the combined organic extracts are washed with a saturated NaCl solution and dried over Na₂SO₄, and the crude reaction product is purified by chromatography on a silica column (DCM then 2% MeOH in DCM). 5 g of the expected product are obtained.

MH+=274.3

### Stage 2:

### 4-[4-(4-(Trifluoromethyl)phenylamino)pyrimidin-2-ylamino]benzoic Acid Methyl Ester

Just as in stage 2 of Procedure 1, starting from 4.6 g of the chloropyrimidine obtained in stage 1 and 2.6 g of methyl 4-aminobenzoate, 6.4 g of the expected product are thus obtained.

### Stage 3:

### 4-[4-(4-(Trifluoromethyl)phenylamino)pyrimidin-2-ylamino]benzoic Acid

Just as in stage 3 of Procedure 1, starting from 6.4 g of the ester obtained in stage 2 and 2.26 g of sodium hydroxide, 4.2 g of the expected product are thus obtained.

MH+=375.1

### EXAMPLE 1

### 4-[4-(4-Fluoro-3-methylphenylamino)pyrimidin-2-ylamino]-N-methyl-N-(1-methylpiperidin-4-yl)benzamide

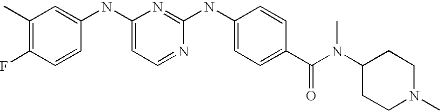

A mixture comprising 470 mg of the acid obtained in Procedure 1 and 230 μl of methyl(1-methylpiperidin-4-yl)-amine is reacted for 3 hours at ambient temperature in the presence of 610 mg of BOP and 700 μl of DIPEA in 15 ml of DCM. The mixture is evaporated to dryness, a 10% potassium carbonate solution is added and extraction is carried out with ethyl acetate. After washing the organic phase with water and drying over Na₂SO₄, a chromatographic separation is carried out on a silica column using, as eluent, DCM/MeOH (88/12; v/v). Evaporation is carried out to dryness and the residue is recrystallized from a DCM/iPr₂O mixture in order to obtain 289 mg of the expected product.

MH+=449.2

M.p.=88° C.

¹ H NMR (DMSO): 1.57 (m, 2), 1.81 (m, 4), 2.14 (1s, 3), 2.25 (s, 3), 2.74-2.94 (unresolved peak, 5), 4.03 (1s, 1), 6.21 (d, 1), 7.09 (t, 1), 7.25 (d, 2), 7.46 (m, 1), 7.59 (d, 1), 7.77 (d, 2), 8.03 (d, 1), 9.33 (s, 1), 9.37 (s, 1)

### EXAMPLE 2

### 4-[4-(4-Fluoro-3-methylphenylamino)pyrimidin-2-ylamino]-N-methyl-N-[1-(4,4,4-trifluorobutyl)piperidin-3-yl]benzamide

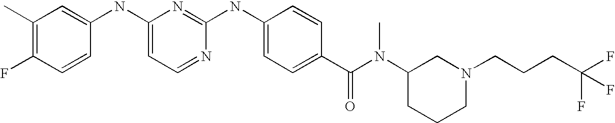

### Stage 1:

### 3-({4-[4-(4-Fluoro-3-methylphenylamino)pyrimidin-2-ylamino]benzoyl}(methyl)amino)piperidine-1-carboxylic Acid Tert-Butyl Ester

According to the procedure described in Example 1, starting from 1.3 g of the acid obtained in Procedure 1 and 800 mg of 3-(methylamino)piperidine-1-carboxylic acid tert-butyl ester, 1.1 g of the expected product are obtained.

### Stage 2:

### 4-[4-(4-Fluoro-3-methylphenylamino)pyrimidin-2-ylamino]-N-methyl-N-(piperidin-3-yl)benzamide

1.05 g of the product obtained in stage 1 are dissolved in 5 ml of MeOH. 15 ml of 2N ethereal hydrochloric acid are added at ambient temperature and the mixture is left stirring overnight. The evaporation is carried out several times in the presence of DCM. 940 mg of the expected piperidine hydrochloride were obtained.

### Stage 3:

### 4-[4-(4-Fluoro-3-methylphenylamino)pyrimidin-2-ylamino]-N-methyl-N-[1-(4,4,4-trifluorobutyl)piperidin-3-yl]benzamide

The hydrochloride obtained in stage 2 (300 mg) is mixed with 90 mg of 4,4,4-trifluorobutyraldehyde and 250 mg of NaBH(OAc)₃ in 10 ml of THF. After reacting overnight, the mixture is taken up in a sodium hydroxide solution, extraction is carried out with DCM and the organic phase is washed and dried over Na₂SO₄. A chromatographic separation is carried out on a silica column using, as eluent, CH₂Cl₂/CH₃OH (99/1, v/v). Evaporation is carried out to dryness and the residue is recrystallized from a CH₂Cl₂/isopropyl ether mixture in order to obtain 141 mg of the expected product.

MH+=545.3

M.p.=100-110° C.

¹H NMR (DMSO): 1.62-2.09 (unresolved peak, 6), 2.24 (s, 3), 2.38 (m, 2), 2.89 (s, 3), 2.96 (m, 1), 3.16-3.33 (2m, 3), 3.49 (m, 2), 4.47 (m, 1), 6.48 (d, 1), 7.13 (t, 1), 7.37 (m, 1), 7.42 (d, 2), 7.49 (m, 1), 7.59 (d, 2), 8.00 (d, 1)

### EXAMPLE 3

### 4-[4-(4-Fluoro-3-methylphenylamino)pyrimidin-2-ylamino]-N-methyl-N-[1-(1,2,3-thiadiazol-4-ylmethyl)piperidin-3-yl]benzamide

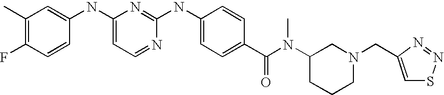

According to the procedure described in stage 3 of Example 2, starting from 330 mg of the hydrochlorate obtained in stage 2 of Example 2 and 100 mg of 1,2,3-thiadiazole-4-carbaldehyde, 191 mg of the expected product are obtained.

MH+=533.2

M.p.=125-130° C.

¹H NMR (DMSO) : 1.08-2.04 (unresolved peak, 5), 2.23 (s, 4), 2.63-2.99 (unresolved peak, 5), 3.46-4.66 (unresolved peak, 3), 6.22 (d, 1), 7.08 (t, 1), 7.22 (m, 2), 7.47 (m, 1), 7.58 (d, 1), 7.64 (d, 2), 8.04 (d, 1), 9.03 (1s, 1), 9.34 (s, 1), 9.37 (s, 1)

### EXAMPLE 4

### N-Methyl-N-(1-methylpiperidin-4-yl)-4-[4-(4-(trifluoromethyl)phenylamino)pyrimidin-2-ylamino]benzamide

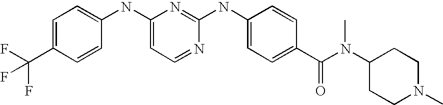

According to the procedure described in Example 1, starting from 400 mg of the acid obtained in Procedure 2 and 130 mg of methyl(1-methylpiperidin-4-yl)amine, 285 mg of the expected product are obtained.

MH+=485.0

M.p.=238-244° C.

¹H NMR (DMSO): 1.59 (m, 2), 1.77-1.93 (unresolved peak, 4), 2.15 (s, 3), 2.82 (d, 2), 2.85 (s, 3), 3.86 (bs, 1), 6.36 (d, 1), 7.28 (d, 2), 7.61 (d, 2), 7.77 (d, 2), 7.92 (d, 2), 8.13 (d, 1), 9.12 (s, 1), 9.52 (s, 1).

### EXAMPLE 5

### N-Methyl-N-(tetrahydropyran-4-yl)-4-[4-(4-(trifluoromethyl)phenylamino)pyrimidin-2-ylamino]benzamide

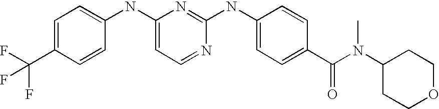

According to the procedure described in Example 1, starting from 400 mg of the acid obtained in Procedure 2 and 115 mg of methyl(tetrahydropyran-4-yl)amine, 288 mg of the expected product are obtained.

MH+=471.9

M.p.=254-256° C.

¹H NMR (DMSO): 1.56 (bd, 2), 1.81 (m, 2), 2.83 (s, 3), 3.25 (bs, 2), 3.88 (bd, 2), 4.13 (bs, 1), 6.34 (d, 1), 7.32 (d, 2), 7.63 (d, 2), 7.80 (d, 2), 7.97 (d, 2), 8.14 (d, 1), 9.51 (s, 1), 9.83 (s, 1).

### EXAMPLE 6

### N-(1-Methylpiperidin-4-yl) -N-(2-(pyrrolidin-1-yl)ethyl)-4-[4-(4-(trifluoromethyl)phenylamino)pyrimidin-2-ylamino]benzamide

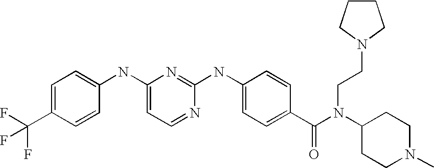

### Stage 1:

### (1-Methylpiperidin-4-yl)(2-(pyrrolidin-1-yl)ethyl)amine

As in stage 3 of Example 2, starting from 3 ml of 1-methylpiperidin-4-one and 3.35 ml of 2-(pyrrolidin-1-yl)ethylamine, 4.4 g of the expected product are obtained.

### Stage 2:

### (1-Methylpiperidin-4-yl)(2-(pyrrolidin-1-yl)ethyl)carbamic Acid Tert-Butyl Ester

A mixture comprising 4.4 g of the compound obtained in stage 1 is dissolved in 100 ml of dichloromethane. 4.7 g of BOC₂O are added to the reaction medium and the mixture is heated at 50° C. for 1 h 30. After concentrating to dryness, the crude product is purified on an alumina column (dichloromethane as a gradient up to 2% of methanol). In total, 2.35 g of the expected compound are obtained.

### Stage 3:

### (1-Methylpiperidin-4-yl)(2-(pyrrolidin-1-yl)ethyl)amine Hydrochloride

According to the decarboxylation reaction described in stage 2 of Example 2, starting from 1.85 g of the amine obtained in stage 2, 1.65 g of the expected aminopiperidine are obtained.

### Stage 4:

### N-(1-Methylpiperidin-4-yl)-N-(2-(pyrrolidin-1-yl)ethyl)-4-[4-(4-(trifluoromethyl)phenylamino)pyrimidin-2-ylamino]benzamide

According to the procedure described in Example 1, starting from 400 mg of the acid obtained in Procedure 2 and 310 mg of the aminopiperidine obtained in stage 4, 44 mg of the expected product are obtained.

MH+=558.1

M.p.=115-120° C.

¹H NMR (DMSO): 1.52-1.96 (unresolved peak, 10), 2.12 (s, 3), 2.43 (unresolved peak, 4), 2.56 (t, 2), 2.78 (d, 2), 3.38 (t, 2), 3.68 (m, 1), 6.35 (d, 1), 7.25 (d, 2), 7.66 (d, 2), 7.77 (d, 2), 7.94 (d, 2), 8.13 (d, 1), 9.24 (s, 1), 9.62 (s, 1)

### EXAMPLE 7

### 4-({4-[(4-Fluorophenyl)amino]pyrimidin-2-yl}amino)-N-(octahydroindolinizin-7-yl)benzamide

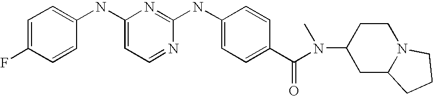

### Stage 1:

### Hexahydroindolizin-7(1H)-one

According to the procedure described in J. Chem. Soc. Perkin Trans. I, 1986, 447-453, 3.6 ml of but-3-en-2-one are added dropwise under cold conditions to a mixture comprising 6 ml of 4,4-diethoxy-1-butamine in 20 ml of Et₂O. The reaction medium is left stirring at AT for 1 h. The reaction medium is decanted into 50 ml of a 2.5M HCl solution and extracted with ether. The aqueous phase is left under warm conditions for 3 h. After cooling and evaporating to dryness, the crude product is taken up in H₂O/K₂CO₃ and extracted with DCM, and the DCM extract is dried and concentrated.

The expected product is obtained by vacuum distillation (40° C., 0.3 mmHg). 1.5 g of the expected product are obtained.

### Stage 2:

### N-Methyloctahydroindolizin-7-amine

By a reduction amination reaction, starting with 1.5 g of the ketone obtained in stage 1, 10.7 ml of a 2M solution of methylamine in THF and 3 g of NaBH (OAc)₃ in 20 ml of THF, the reaction medium is heated at 60° C. for 1 h. After evaporating, taking out the residue in H₂O/NaOH and extracting with DCM, the DCM extract is dried and, after concentrating to dryness, 1.25 g of the expected amine are obtained.

### Stage 3:

### 4-({4-[(4-Fluorophenyl)amino]pyrimidin-2-yl}amino)-N-(octahydroindolinizin-7-yl)benzamide

According to the procedure described in Example 1, starting from 600 mg of the acid obtained in Procedure 1 and 255 mg of N-methyloctahydroindolizin-7-amine, 172 mg of the expected benzamide are obtained.

MH+=461.1

M.p.=230-235° C.

¹H NMR (DMSO): 1.2-2.14 (unresolved peak, 11), 2.84 (s, 3), 2.92 (t, 1), 3.03 (m, 1), 3.96 (m, 1), 6.23 (d, 1), 7.13 (t, 2), 7.25 (d, 2), 7.65 (m, 2), 7.77 (d, 2), 8.04 (d, 1), 9.04 (s, 1), 9.15 (s, 1).

### EXAMPLE 8

### Pharmaceutical Composition

Tablets were prepared corresponding to the following formulation:

| | | |
|---|---|---|
| Product of Example 1 | 0.2 | g |
| Excipient for a tablet made up to | 1 | g |
| (breakdown of the excipient: lactose, | | |
| talc, starch, magnesium stearate). | | |

Example 1 is taken as example in the pharmaceutical preparation constituted by Example 8 above, it being possible for this pharmaceutical preparation to be produced differently as indicated above and if desired with other products in examples in the present patent application.

### Pharmacological Part:

### Protocols for Biochemical Trials on IKK

### I) Evaluation of the Compounds With Regard to IKK1 and IKK2:

The compounds are tested for inhibition of IKK1 and IKK2 using a kinase test on a flash plate support. The test compounds are dissolved at 10 mM in DMSO and then diluted in kinase buffer (50 mM Tris, pH 7.4, containing 0.1 mM EGTA, 0.1 mM sodium orthovanadate and 0.1% p-mercaptoethanol).

Serial three-fold dilutions are carried out starting from this solution. 10 μl of each dilution are added to the wells of a 96-well plate in duplicate. 10 μl of kinase buffer are added to the control wells, which will serve for 0% inhibition, and 10 μl of 0.5 mM EDTA are added to the control wells (100% inhibition). 10 μl of the mixture IKK1 or IKK2 (0.1 μg/well), biotinylated 25-55 IKB substrate peptide and BSA (5 μg) are added to each well. To initiate the kinase reaction, 10 μl of the mixture of 10 mM magnesium acetate, 1 μM cold ATP and 0.1 μCi³³P-ATP are added to each well for a final volume of 30 μl. The reaction is then incubated at 30° C. for 90 min and then halted by the addition of 40 μl of 0.5 mM EDTA. After stirring, 50 μl are transferred to a flash plate covered with streptavidin.

30 min later, the wells are washed twice with a 50 mM Tris-EDTA, pH 7.5, solution and the radioactivity is determined on a MicroBeta counter.

The compounds of the invention tested in this trial show an IC₅₀ of less than 10 μM, which shows that they can be used for their therapeutic activity.

### II) Evaluation of the Compounds With Regard to the Viability and the Proliferation of Tumor Cells:

The compounds according to the invention formed the subject of pharmacological trials which make it possible to determine their anticancer activity.

The compounds of formula (I) according to the present invention were tested in vitro on a sample group of tumor lines of human origin originating:
from breast cancer: MDA-MB231 (American Type Culture Collection, Rockville, Md., USA, ATCC-HTB26), MDA-A1 or MDA-ADR (referred to as multidrug resistant MDR line, and described by E.Collomb et al. in Cytometry, 12(1), 15-25, 1991), and MCF7 (ATCC-HTB22),
from prostate cancer: DU145 (ATCC-HTB81) and PC3 (ATCC-CRL1435),
from colon cancer: HCT116 (ATCC-CCL247) and HCT15 (ATCC-CCL225),
from lung cancer: H460 (described by Carmichael in Cancer Research, 47 (4), 936-942, 1987, and provided by the National Cancer Institute, Frederick Cancer Research and Development Center, Frederick, Md., USA),
from glioblastoma: SF268 (described by Westphal in Biochemical & Biophysical Research Communications, 132 (1), 284-289, 1985, and provided by the National Cancer Institute, Frederick Cancer Research and Development Center, Frederick, Md., USA),
from leukemia: CMLT1 (described by Kuriyama et al. in Blood, 74, 1989, 1381-1387, by Soda et al. in British Journal of Haematology, 59, 1985, 671-679, and by Drexler in Leukemia Research, 18: 1994, 919-927, and provided by DSMZ, Mascheroder Weg 1b, 38124, Braunschweig, Germany).

The cell proliferation and viability were determined in a test using 3-(4,5-dimethylthiazol-2-yl)-5-(3-carboxymethoxyphenyl)-2-(4-sulphophenyl)-2H-tetrazolium (MTS) according to Fujishita T. et al., Oncology, 2003, 64 (4), 399-406. In this test, the mitochondrial ability of the living cells to convert MTS to a colored compound is measured after incubating for 72 hours a compound of formula (I) according to the invention. The concentrations of compound according to the invention which result in a 50% loss of cell proliferation and viability (IC₅₀) are less than 10 μM, depending on the tumor line and the compound tested.

Thus, according to the present invention, it appears that the compounds of formula (I) bring about a loss of proliferation and viability of tumor cells with an IC₅₀ of less than 10 μM.

What is claimed is:

1) A compound of formula (I): 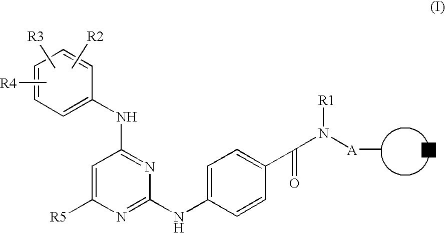
wherein:
R2, R3 and R4, which are identical or different, are such that one represents a halogen atom or CF₃, and the other two, which are identical or different, represent a hydrogen, halogen, or an alkyl radical or an alkoxy radical optionally substituted by one or more halogen atoms;
R5 represents a hydrogen or halogen;
R1 represents a hydrogen, a cycloalkyl radical or an alkyl, alkenyl or alkynyl radical, all optionally substituted by one or more identical or different radicals chosen from halogen, OR8 and NR8R9, the alkyl radical represented by R1 in addition is optionally substituted by a saturated or unsaturated 5-membered heterocyclic radical attached via a carbon atom and optionally substituted by one or more radicals chosen from one or more halogen, alkyl or alkoxy radicals;
A represents a single bond or a —CH₂—CO—NR6— radical, and R6, which is identical to or different from R1, is chosen from the values of R1;
the ring comprising Y is monocyclic or bicyclic, has from 4 to 10 ring members and is saturated or partially saturated, wherein Y represents an oxygen atom, a sulphur atom optionally oxidized by one or two oxygen atoms,
N—R7, C═O or its dioxolane as protective group for the carbonyl functional group, CF₂, CH—OR8 or CH—NR8R9; and
wherein when Y represents NR7, the ring comprising Y can include a carbon bridge composed of 1 to 3 carbons;
R7 represents hydrogen, a cycloalkyl radical or an alkyl, CH₂-alkenyl or CH₂-alkynyl radical, all optionally substituted by a naphthyl radical or by one or more identical or different radicals chosen from halogen, hydroxyl, alkoxy, phenyl and heteroaryl radicals, the alkyl radical represented by R7 in addition is optionally substituted by a hydroxyl, —NR8R9, —CO-NR8R9, phosphonate, alkylthio, optionally oxidized to give sulphone, or optionally substituted heterocycloalkyl radical;
R8 represents hydrogen, alkyl, cycloalkyl or heterocycloalkyl, which radicals are optionally substituted by one or more radicals chosen from halogen, hydroxyl, alkoxy, NH₂, NHalkyl, N(alkyl)₂, —CONH₂, —CONHalkyl or
—CON(alkyl)₂, the alkyl radical represented by R8 in addition is optionally substituted by an alkylthio radical, by an optionally substituted phenyl radical or by a saturated or unsaturated, optionally substituted, heterocyclic radical;
NR8R9 is such that R8 and R9, which are identical or different, are chosen from the values of R8,
or R8 and R9 form, with the nitrogen atom to which they are bonded, an optionally substituted cyclic amine which optionally contains one or two other heteroatoms chosen from O, S, N and NR10; and wherein
all of the above heterocyclic, heterocycloalkyl and heteroaryl radicals are composed of 4 to 10 ring members, unless specified, and include 1 to 4 heteroatoms chosen from O, optionally oxidized S, N and NR10; and wherein
all of the above naphthyl, phenyl, heterocyclic, heterocycloalkyl and heteroaryl radicals and also the cyclic amine which can be formed by R8 and R9 with the nitrogen atom to which they are bonded, are optionally substituted by one or more identical or different radicals chosen from halogen, hydroxyl, alkoxy, alkyl, hydroxyalkyl, alkoxyalkyl, CN, CF₃, NH₂, NHalk and N(alk)₂; and
R10 represents a hydrogen or an alkyl;
or an acid addition salt thereof.

2) The compound of formula (I) according to claim, 1 wherein:
R1 represents hydrogen or a linear or branched alkyl radical having from 1 to 5 carbon atoms optionally substituted by an optionally substituted saturated or unsaturated heterocycle;
or an acid addition salt thereof.

3) The compound of formula (I) according to claim 1, wherein:
R1 represents hydrogen or an optionally substituted, linear or branched, alkyl radical having from 1 to 4 carbon atoms; and
Y represents NR7, wherein R7 represents a linear or branched alkyl radical having from 1 to 6 carbon atoms which is optionally substituted by a radical chosen from hydroxyl, CF₃, phosphonate, sulphone, optionally substituted phenyl and optionally substituted, saturated or unsaturated, monocyclic or bicyclic, heterocyclic radicals;
or an acid addition salt thereof.

4) The compound of formula (I) according to claim 3, wherein R1 represents a CH₃.

5) The compound of formula (I) according to claim 1, wherein:
R1 represents a linear or branched alkyl radical having from 1 to 4 carbon atoms; and
Y represents CH—NR8R9, wherein R8 represents a hydrogen or CH₃ and R9 represents a linear or branched alkyl radical having from 1 to 6 carbon atoms which is optionally substituted by a radical chosen from hydroxyl, CF₃, optionally substituted phenyl and optionally substituted saturated or unsaturated, monocyclic or bicyclic, heterocyclic radical,
or an acid addition salt thereof.

6) The compound of formula (I) according to claim 1,
R1 represents hydrogen, a cycloalkyl radical or an alkyl, alkenyl or alkynyl radical, all optionally substituted by one or more identical or different radicals chosen from halogen atoms, OR8 and NR8R9;
Y represents an oxygen atom, a sulphur atom optionally oxidized by one or two oxygen atoms, N—R7, C═O, CF₂, CH—OR8 or CH—NR8R9;
R7 represents a hydrogen or an alkyl, CH₂-alkenyl or CH₂-alkynyl radical, all optionally substituted by a naphthyl radical or by one or more identical or different radicals chosen from halogen, hydroxyl, phenyl and heteroaryl radicals, wherein the naphthyl, phenyl and heteroaryl radicals are optionally substituted; the heteroaryl radicals is composed of 5 to 10 ring members and has 1 to 4 heteroatoms chosen from O, S, N and NR10;
R8 represents hydrogen or alkyl, cycloalkyl or heterocycloalkyl radicals, which radicals are optionally substituted by one or more radicals chosen from halogen, hydroxyl, alkoxy, NH₂, NHalkyl or N(alkyl)₂;
NR8R9 is such that R8 and R9, which are identical or different, are chosen from the values of R8;
or R8 and R9 form, with the nitrogen atom to which they are bonded, a cyclic amine which optionally contains one or two other heteroatoms chosen from O, S, N and optionally substituted NR10;
R10 represents a hydrogen atom or an alkyl radical; and wherein
all of the naphthyl, phenyl and heteroaryl radicals and also the cyclic amine which can be formed by R8 and R9 with the nitrogen atom to which they are bonded are optionally substituted by one or more identical or different radicals chosen from halogen, hydroxyl, alkoxy, alkyl, hydroxyalkyl, alkoxyalkyl, CF₃, NH₂, NHalk and N(alk)₂;
or an acid addition salt thereof.

7) The compound of formula (I) according to claim 1, wherein:
R2, R3 and R4, which are identical or different, are such that one represents a fluorine or chlorine atom or CF₃ and the other two, which are identical or different, represent hydrogen, fluorine, or chlorine or a methyl or methoxy radical optionally substituted by one or more fluorine atoms;
R5 represents hydrogen, fluorine, or chlorine;
R1 represents hydrogen, cycloalkyl or an alkyl radical optionally substituted by one or more identical or different radicals chosen from the fluorine atom, OR8 and NR8R9;
A represents a single bond or a —CH₂—CO—NR6— radical and R6 represents a hydrogen atom or a linear or branched alkyl radical including at most 4 carbon atoms;
Y represents an oxygen atom, a sulphur atom optionally oxidized by one or two oxygen atoms, N—R7, C═O, CF₂, CH—OR8 or CH—NR8R9;
R7 represents hydrogen or a alkyl radical optionally substituted by one or more identical or different radicals chosen from halogen, phenyl and heteroaryl, the phenyl and heteroaryl radicals are optionally substituted by one or more identical or different radicals chosen from halogen, hydroxyl, alkoxy, alkyl, hydroxyalkyl, alkoxyalkyl, CF₃, NH₂, NHalk and N(alk)₂ radicals; and wherein
the heteroaryl radicals are composed of 5 to 7 ring members and include 1 to 3 heteroatoms chosen from O, S, N and NR10;
R8 represents hydrogen, a linear or branched alkyl radical having at most 4 carbon atoms or a cycloalkyl radical having from 3 to 6 ring members, wherein the alkyl and cycloalkyl radicals are optionally substituted by a hydroxyl radical;
NR8R9 is such that R8 and R9, which are identical or different, are chosen from the values of R8;
or R8 and R9 form, with the nitrogen atom to which they are bonded, a cyclic amine chosen from pyrrolyl, piperidyl, morpholinyl, pyrrolidinyl, azetidinyl and piperazinyl radicals, wherein the piperazinyl radical is optionally substituted on its second nitrogen atom by an alkyl radical;
or an acid addition salt thereof.

8) The compound of formula (I) according to claim 1, wherein:
R2, R3 and R4, which are identical or different, are such that one represents a fluorine or CF₃, one represents a hydrogen atom, and one represents a fluorine, chlorine, or ethyl;
R5 represents a hydrogen or a chlorine;
R1 represents a hydrogen atom or a cyclopropyl, methyl, ethyl, propyl or butyl radical optionally substituted by one or more identical or different radicals chosen from fluorine, hydroxyl, amino, alkylamino, dialkylamino, piperidinyl, morpholinyl, azetidinyl, piperazinyl, pyrrolidinyl and pyrrolyl;
A represents a single bond, —CH₂—CO—NH— or —CH₂—CO—NCH₃—; the ring comprising Y is chosen from a cyclohexyl which is substituted by amino; tetrahydropyranyl; dioxidothienyl; and pyrrolidinyl, piperidinyl, azepinyl, indolizinyl and quinazolinyl radicals which are optionally substituted by one or more identical or different radicals chosen from methyl, propyl, butyl, isopropyl, isobutyl, isopentyl and ethyl, which radicals are optionally substituted by one or more radicals chosen from halogen, hydroxyl, phenyl optionally substituted by one or more halogen atoms, quinolyl, pyridyl optionally oxidized on its nitrogen atom, thiophenyl, thiazolyl, thiadiazolyl, tetrazolyl, pyrazinyl, furyl and imidazolyl optionally substituted by alkyl;
or an acid addition salt thereof.

9) The compound of formula (I) according to claim 1, wherein:
R2, R3 and R4, which are identical or different, are such that one represents fluorine or CF₃, one represents a hydrogen, and one represents fluorine, chlorine, or methyl;
R5 represents hydrogen;
R1 represents methyl or ethyl optionally substituted by an amino, alkylamino, dialkylamino or pyrrolidinyl;
A represents a single bond; and
the ring comprising Y represents a cyclohexyl radical substituted by amino; or a piperidinyl or pyrrolidinyl radical optionally substituted on the nitrogen atom by a methyl, propyl, butyl, isopropyl, isobutyl, isopentyl or ethyl radical, which radicals are optionally substituted by one or more halogen atoms or a radical chosen from hydroxyl; thiadiazolyl; tetrazolyl; phenyl optionally substituted by halogen; quinolyl; pyridyl optionally oxidized on its nitrogen atom; furyl; and imidazolyl optionally substituted by alkyl;
or an acid addition salt thereof.

10) The compound of formula (I) according to claim 1, wherein:
R2, R3 and R4, which are identical or different, are such that one represents fluorine or CF₃, one represents hydrogen, and one represents fluorine, chlorine, or methyl;
R5 represents a hydrogen atom;
R1 represents hydrogen or methyl;
A represents a single bond; and
the ring comprising Y is chosen from tetrahydropyranyl; dioxidothiophenyl; pyrrolidinyl, piperidinyl and azepinyl radicals optionally substituted on their nitrogen atoms in the 2 or 3 position of the ring by a methyl, ethyl, propyl or butyl radical, which radicals are optionally substituted by one or more halogen, phenyl, pyridyl, thiophenyl, thiazolyl, thiadiazolyl, pyrazinyl, furyl or imidazolyl;
or an acid addition salt thereof.

11) The compound of formula (I) according to claim 1 selected from the group consisting of:
4-[4-(4-fluoro-3-methylphenylamino)pyrimidin-2-ylamino]-N-methyl-N-(1-methylpiperidin-4-yl)benzamide;
4-[4-(4-fluoro-3-methylphenylamino)pyrimidin-2-ylamino]-N-methyl-N-[1-(4,4,4-trifluorobutyl)piperidin-3-yl]benzamide;
4-[4-(4-fluoro-3-methylphenylamino)pyrimidin-2-ylamino]-N-methyl-N-[1-(1,2,3-thiadiazol-4-ylmethyl)piperidin-3-yl]benzamide;
N-methyl-N-(1-methylpiperidin-4-yl)-4-[4-(4-(trifluoromethyl)phenylamino)pyrimidin-2-ylamino]benzamide;
N-methyl-N-(tetrahydropyran-4-yl)-4-[4-(4-(trifluoromethyl)phenylamino)pyrimidin-2-ylamino]benzamide;
N-(1-methylpiperidin-4-yl)-N-[2-(pyrrolidin-1-yl)ethyl]-4-[4-(4-(trifluoromethyl)phenylamino)pyrimidin-2-ylamino]benzamide; and
4-({4-[(fluorophenyl)amino]pyrimidin-2-yl}amino)-N-(octahydroindolinizin-7-yl)benzamide;
or an acid addition salt thereof.

12) A process for the preparation of the compound of formula (I) as defined in claim 1, said process comprising reacting the compound of formula (II) wherein R₅′ is as defined in claim 1 for R5 in which the possible reactive functions are optionally protected: 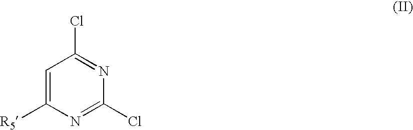
with a compound of formula (III): 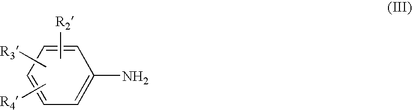
wherein R₂′, R₃′ and R₄′ are as defined in claim 1 for R2, R3 and R4, respectively, in which the possible reactive functional groups are optionally protected,
in order to obtain a compound of formula (IV): 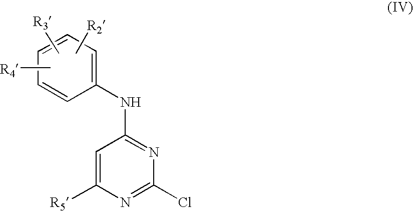
in which R₂′, R₃′, R₄′ and R₅′ are as defined above;
reacting the compound of formula (IV) with the methyl ester of 4-aminobenzoic acid, in order to obtain the compound of formula (VI): 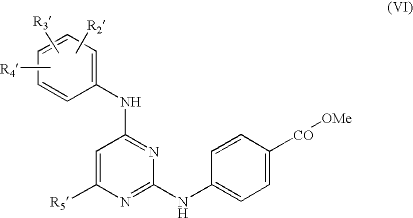
wherein R₂′, R₃′, R₄′ and R₅′ are as above;
saponifying the compound of formula (VI) to give its corresponding acid of formula (VII): 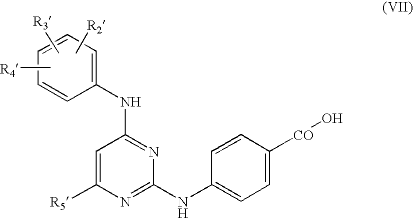
wherein R₂′, R₃′, R₄′ and R₅′are as defined above;
reacting the compound of formula (VII) with an amine of formula (VIII): 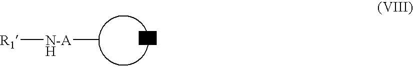
wherein A and the ring comprising Y are as defined in claim 1, and R₁′ is as defined in claim 1 for R1 in which the possible reactive functional groups are optionally protected by protective groups,
in order to obtain a compound of the formula (I₁): 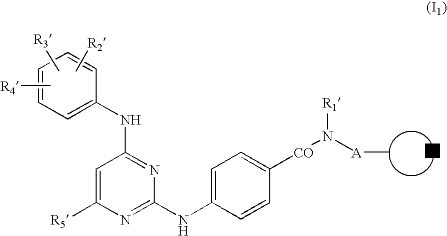
wherein A, Y, R₁′, R₂′, R₃′, R₄′ and R₅′ are as defined above, and wherein the compound of formula (I₁) can be the compound of formula (I) or, in order to obtain the compound of formula (I), can be subjected to one or more of the following conversion reactions, in any order:
a) an oxidation reaction on an alkylthio group to give the corresponding sulphoxide or sulphone,
b) a conversion reaction on an alkoxy functional group to give a hydroxyl functional group or also on a hydroxyl functional group to give an alkoxy functional group,
c) an oxidation reaction on an alcohol functional group to give an aldehyde or ketone functional group,
d) an elimination reaction on the protective groups which can be carried by the protected reactive functional groups,
e) a salification reaction by an inorganic or organic acid in order to obtain the corresponding salt, and
f) a resolution reaction on the racemic forms to give resolved products.

13) A process for the preparation of the compound of formula (I) as defined in claim 1, said process comprising subjecting the compound of formula (A) wherein ring(N) is as defined in claim 1, and R₁′, R₂′, R₃′, R₄′, and R₅′ are as defined in claim 1 for R2, R3, R4, and R5, respectively, in which the possible reactive functional groups are optionally protected: 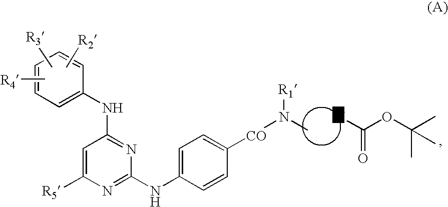
to a deprotection reaction on the carbamate functional group, in order to obtain a compound of formula (IX): 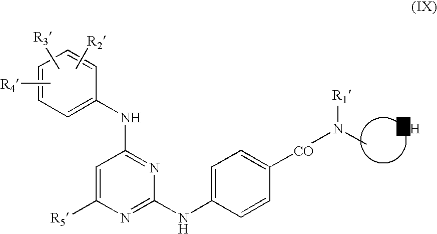
wherein R₁′, R₂′, R₃′, R₄′, R₅′ and ring(N) are as defined above;
subjecting the compound of formula (IX) to reductive amination conditions in the presence of an aldehyde or ketone of formula (X) in which RZ′ represents an optionally substituted alkyl, alkenyl or alkynyl radical as defined in claim 1 and in which the possible reactive functional groups are optionally protected by protective groups, and wherein R8′ is as defined in claim 1 for R8 in which the possible reactive functional groups are optionally protected by protective groups:
RZ′—CR8′O (X),
in order to obtain a compound of formula (I₂): 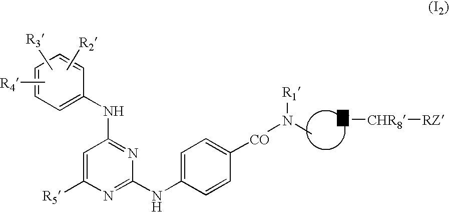
wherein R₁′, R₂′, R₃′, R₄′, R₅′, ring(N), RZ′ and R8′ are as defined above,
and wherein the compound of formula (I₂) can be a compound of formula (I) or, in order to obtain a compound of formula (I), can be subjected to one or more of the following conversion reactions, in any order:
a) an oxidation reaction on an alkylthio group to give the corresponding sulphoxide or sulphone,
b) a conversion reaction on an alkoxy functional group to give a hydroxyl functional group or also on a hydroxyl functional group to give an alkoxy functional group,
c) an oxidation reaction on an alcohol functional group to give an aldehyde or ketone functional group,
d) an elimination reaction on the protective groups which can be carried by the protected reactive functional groups,
e) a salification reaction by an inorganic or organic acid in order to obtain the corresponding salt, and
f) a resolution reaction on the racemic forms to give resolved products.

14) A pharmaceutical composition comprising a compound of formula (I) according to claim 1, or a pharmaceutically acceptable salt thereof and a pharmaceutically acceptable vehicle.

15) A pharmaceutical composition comprising a compound of formula (I) according to claim 11, or a pharmaceutically acceptable salt thereof and a pharmaceutically acceptable vehicle.

16) A method of inhibiting the activity of the protein kinase IKK comprising contacting said protein kinase with a compound according to claim 1 or a pharmaceutically acceptable salt thereof.

17) The method according to claim 16 wherein the protein kinase is in a mammal.

18) A method of treating or preventing a disease selected from inflammatory diseases, diabetes and cancer, comprising administering to a patient in need of said treatment or prevention a therapeutically effective amount of compound according to claim 1 or a pharmaceutically acceptable salt thereof.

19) The method according to claim 18 wherein the disease is cancer.

20) The method according to claim 19 wherein the cancer is resistant to cytotoxic agents.
